SEQUENCE LISTING (16 sequences)

SEQ ID NO: 1 - protein, 391 aa, Flavobacterium sp.
NARSTGQHPARYPGAAAGEPTLDSWQEPPHNRWAFAHLGEMVPSAAVSRRPVNAPGHALARLGAIAAQLPDLEQRLEQTYTDAFLVLRGTEVVAEYYRAGFAPDDRHLLMSVSKSLCGTVVGALVDEGRIDPAQPVTEYVPELAGSVYDGPSVLQVLDMQISIDYNEDYVDPASEVQTHGRSAGWRTRRHGDPADTYEFLTTLRGDGSTGEFQYCSANTDVLAWIVERVTGLRYVEALSTYLWAKLDADRDATITVDTTGFGFAHGGVSCTARDLARVGRMMLDGGVAPGGRVVSEDWVRRVLAGGSHEAMTDKGFTNTFPDGSYTRQWWCTGNERGNVSGIGIHGQNLWLDPLTDSVIVKLSSWPDPDTEHWHRLQNGILLDVSRALDAV

SEQ ID NO: 2 - DNA, 1173 bp, Flavobacterium sp.
  source 1..1173: mol_type = other DNA
aacgcgcgttcaaccggtcagcatcctgcccgctatccaggcgcggccgcaggcgagcccaccctcgatagttggcaggaaccaccacataaccgttgggctttcgcgcatttgggtgaaatggtcccatctgctgccgtctcgagacgtccggttaacgctccgggacacgcgttggcccgtttgggagcaattgccgcccagctgccggacttggaacaacgtttggaacagacgtatacagacgcatttctggtattacggggcaccgaagtggtggcggaatattatagagcagggtttgcccctgacgaccgccaccttctgatgagcgtgtcgaaatccctctgtggcaccgtggtgggcgcactcgtcgatgaaggccgtattgatccggcacagccagtcacggaatacgtgcccgagctggcgggtagcgtctatgatggaccctctgttttacaggtcttggatatgcagatttcaattgattacaacgaagactatgttgacccggcgtcagaggttcaaactcacggccggtctgcagggtggcgtacccgccgtcatggggatccggccgacacgtatgaattcctgaccacacttcgtggtgatggctccaccggtgagtttcaatattgctccgccaatacagatgtgctggcctggattgtggagcgtgttaccgggcttcgttatgtagaagccctgtcaacatatttgtgggccaaactggatgcggatcgtgatgcgactatcacagttgataccaccggcttcggtttcgctcatggcggtgtgtcgtgcacagcacgggatctggcccgtgtcggtcgtatgatgttagacggtggggtggcacccgggggtcgcgttgtttcagaagactgggtccgtcgtgttctggctggcggatcacatgaagcaatgacggacaagggctttacaaatacgtttcccgatggctcatatacgcggcaatggtggtgtacaggcaatgagcggggtaatgtttccggaataggtatccacggacagaatttatggctcgatccattaacagacagcgtcatagtcaagttgtcttcttggccggacccagatactgagcactggcaccgcctgcagaatggcatcctgctggatgtcagccgagccctggatgctgtt

SEQ ID NO: 3 - protein, 391 aa, synthetic construct
NARSTGQHPARYPGAAAGEPTLDSWQEPPHNRWAFAHLGEMVPSAAVSRRPVNAPGHALARLGAIAAQLPDLEQRLEQTYTDAFLVLRGTEVVAEYYRAGFAPDDRHLLMSVSKSLCGTVVGALVDEGRIDPAQPVTEYVPELAGSVYDGPSVLQVLDMQISIDYNEDYVDPASEVQTHGRSAGWSTRRHGDPADTYEFLTTLRGDGSTGEFQYCSANTDVLAWIVERVTGLRYVEALSTYLWAKLDADRDATITVDTTGFGCAHGGVSCTARDLARVGRMMLDGGVAPGGRVVSEDWVRRVLAGGSHEAMTDKGFTNTFPDGSYTRQWWCTGNERGNVSGIGIHGQNLWLDPLTDSVIVKLSSWPDPYTEHWHRLQNGILLDVSRALDAV

SEQ ID NO: 4 - DNA, 1173 bp, synthetic construct
  source 1..1173: mol_type = other DNA
aacgcgagaagtaccggccagcatccggcacgctatccgggtgcggccgcaggggaacccacactggatagctggcaagagccaccccataaccgttgggcgttcgcgcatcttggtgaaatggtaccgtcagcagcagtgtcccgacggccggttaacgcgcctggtcatgcgctggcacgcctcggagctatcgcagcgcagctgcctgatctggagcagcgtcttgaacagacatatacagatgcatttttggtacttcgcggcactgaagttgtagcagaatattatcgtgcgggttttgctccggacgaccggcatcttctgatgagtgtttctaaatcgttgtgcgggacagtcgtgggagcactcgtcgacgagggcagaatcgatcctgcacaacctgtcactgaatacgtgcctgaattagccggatcggtatatgatggtccttcagtgctgcaggtcctggacatgcagatttcgatagactacaacgaagactatgttgacccagcgagtgaggtacaaactcatggacgctccgctggctggtccacacgacggcacggtgatcccgctgacacttatgaatttttgacaacactgcgcggcgatggtagtacaggtgaatttcagtactgttcggcgaataccgacgtgcttgcgtggatcgtggaacgcgttaccgggctgagatatgttgaagcgctttctacatacctgtgggccaaattagacgccgatcgtgatgctacaataaccgttgacacaacaggtttcggctgcgcacatggtggtgtttcgtgtacagcccgggatctggctcgcgtaggccggatgatgctggatggcggtgtagccccgggtggtcgagtggtttcagaagattgggttcggcgagtgttagcaggtggaagtcacgaagctatgacggataaaggctttacgaacaccttccctgatggttcttatacacgtcagtggtggtgcacgggtaacgaacgtggcaatgtgagtggtatcggaatccatggtcagaacctgtggctcgatccgctgaccgattcggttattgtgaaactgtcgtcgtggccggacccgtacacagaacattggcatagattgcaaaacggtattctgctggatgtatcacgcgcgttagacgcggtg

SEQ ID NO: 5 - protein, 354 aa, Flavobacterium sp.
NTTPVHALTDIDGGIAVDPAPRLAGPPVFGGPGNDAFDLAPVRSTGREMLRFDFPGVSIGAAHYEEGPTGATVIHIPAGARTAVDARGGAVGLSGGYDFNHAICLAGGASYGLEAGAGVSGALLERLEYRTGFAEAQLVSSAVIYDFSARSTAVYPDKALGRAALEFAVPGEFPQGRAGAGMSASAGKVDWDRTEITGQGAAFRRLGDVRILAVVVPNPVGVIMDRAGTVVRGNYDAQTGVRRHPVFDYQEAFAEQVPPVTEAGNTTISAIVTNVRMSPVELNQFAKQVHSSMHRGIQPFHTDMDGDTLFAVTTDEIDLPTTPGSSRGRLSVNATALGAIASEVMWDAVLEAGK

SEQ ID NO: 6 - DNA, 1062 bp, Flavobacterium sp.
  source 1..1062: mol_type = genomic DNA
aataccacaccggtacacgctcttactgatattgatggaggcatcgcggtggacccagctccacgtcttgcaggacctccagtcttcggaggtccaggtaacgacgcttttgaccttgccccggtccgttctactggtcgtgagatgctgcgcttcgatttcccgggtgttagcatcggcgcggcgcactatgaagaaggtccgactggtgcgaccgtaattcacatcccagctggagcacgtacagctgttgacgcacgtggtggtgcagtgggtctgtccggtggctacgactttaatcacgctatctgcctggcaggtggcgcaagttatggtttagaagctggcgcaggtgtgtctggtgcgctgctggaacgtttggaatatcgtactggctttgcggaagcacagctggttagctctgcagttatttacgatttctctgcgcgctctaccgcagtctatccggataaagctcttggtcgtgcggcgctggagtttgcggttccgggcgaattcccgcagggtcgtgctggtgcgggcatgagtgcttctgcgggtaaagttgattgggaccgtactgaaatcacgggccagggtgctgcgttccgtcgcctgggcgacgttcgcatcctggccgtagttgtcccgaacccggtaggtgtgattatggaccgtgctggcacggtagttcgtggtaactacgatgcacagactggcgttcgtcgtcacccggtattcgattaccaggaagcttttgcggagcaggttcctccggttaccgaagctggtaacaccactatttctgccatcgttaccaacgtacgcatgtcccctgtagagctgaaccaattcgcaaaacaggtacacagctctatgcaccgtggtatccagcctttccacaccgacatggacggtgacaccctgtttgccgtgaccactgacgaaatcgatttgccgacaaccccgggtagctcacgtggtcgcctgtctgtaaacgctaccgctctgggcgcgattgcgtccgaggtcatgtgggacgctgtgctggaagcaggcaag

SEQ ID NO: 7 - protein, 354 aa, synthetic construct
NTTPVHALTDIDGGIAVDPAPRLAGPPVFGGPGNDAFDLAPVRSTGREMLRFDFPGVSIGAAHYEEGPTGATVIHIPAGARTAVDARGGAVGLSGGYDFNHAICLAGGAGYGLEAGAGVSGALLERLEYRTGFAELQLVSSAVIYDFSARSTAVYPDKALGRAALEFAVPGEFPQGRAGAGMSASAGKVDWDRTEITGQGAAFRRLGDVRILAVVVPNPVGVIMDRAGTVVRGNYDAQTGVRRHPVFDYQEAFAEQVPPVTQAGNTTISAIVTNVRMSPVELNQFAKQVHSSMHRGIQPFHTDMDGDTLFAVTTDEIDLPTTPGSSRGRLSVNATALGAIASEVMWDAVLEAGK

SEQ ID NO: 8 - DNA, 1062 bp, synthetic construct
  source 1..1062: mol_type = other DNA
aataccacaccggtacacgctcttactgatattgatggaggcatcgcggtggacccagctccacgtcttgcaggacctccagtcttcggaggtccaggtaacgacgcttttgaccttgccccggtccgttctactggtcgtgagatgctgcgcttcgatttcccgggtgttagcatcggcgcggcgcactatgaagaaggtccgactggtgcgaccgtaattcacatcccagctggagcacgtacagctgttgacgcacgtggtggtgcagtgggtctgtccggtggctacgactttaatcacgctatctgcctggcaggtggcgcaggttatggtttagaagctggcgcaggtgtgtctggtgcgctgctggaacgtttggaatatcgtactggctttgcggaactacagctggttagctctgcagttatttacgatttctctgcgcgctctaccgcagtctatccggataaagctcttggtcgtgcggcgctggagtttgcggttccgggcgaattcccgcagggtcgtgctggtgcgggcatgagtgcttctgcgggtaaagttgattgggaccgtactgaaatcacgggccagggtgctgcgttccgtcgcctgggcgacgttcgcatcctggccgtagttgtcccgaacccggtaggtgtgattatggaccgtgctggcacggtagttcgtggtaactacgatgcacagactggcgttcgtcgtcacccggtattcgattaccaggaagcttttgcggagcaggttcctccggttacccaagctggtaacaccactatttctgccatcgttaccaacgtacgcatgtcccctgtagagctgaaccaattcgcaaaacaggtacacagctctatgcaccgtggtatccagcctttccacaccgacatggacggtgacaccctgtttgccgtgaccactgacgaaatcgatttgccgacaaccccgggtagctcacgtggtcgcctgtctgtaaacgctaccgctctgggcgcgattgcgtccgaggtcatgtgggacgctgtgctggaagcaggcaag

SEQ ID NO: 9 - protein, 354 aa, Flavobacterium sp.
NTTPVHALTDIDGGIAVDPAPRLAGPPVFGGPGNDAFDLAPVRSTGREMLRFDFPGVSIGAAHYEEGPTGATVIHIPAGARTAVDARGGAVGLSGGYDFNHAICLAGGAGYGLEAGAGVSDALLERLEHRTGFAELQLVSSAVIYDFSARSTAVYPDKALGRAALEFAVPGEFPQGRAGAGMSASAGKVDWDRTEITGQGAAFRRLGDVRILAVVVPNPVGVIVDRAGTVVRGNYDAQTGVRRHPVFDYQEAFAEQVPPVTEAGNTTISAIVTNVRMSPVELNQFAKQVHSSMHRGIQPFHTDMDGDTLFAVTTDEIDLPTTPGSSRGRLSVNATALGAIASEVMWDAVLEAGK

SEQ ID NO: 10 - DNA, 1062 bp, Flavobacterium sp.
  source 1..1062: mol_type = genomic DNA
aataccacaccggttcacgcgctgaccgatatcgacggtggtattgcagtcgatccagctccacgtctagctggtcctccagttttcggtggtccgggcaacgatgcttttgacctggcaccggttcgtagcaccggtcgtgaaatgctgcgttttgatttcccgggcgtcagcattggtgccgctcactatgaagaaggtccgaccggtgctaccgtaattcatatcccggcaggcgcccgcactgccgttgacgctcgtggtggtgctgtaggcctgtccggcggctatgattttaaccacgctatttgtctggctggcggcgcaggttatggcctggaagcgggtgcgggtgtaagcgatgctctgctggaacgtctggagcaccgtaccggtttcgcagaattacagctggtgtcttcggctgtgatttacgacttctccgcgcgttccactgccgtttacccggataaagcgctcggccgtgcagcattggaattcgcggtaccgggtgaattcccgcagggtcgtgcgggcgctggcatgtccgcttccgctggtaaggtagactgggatcgtaccgagatcaccggtcagggtgcagctttccgtaggctgggtgacgtgcgtatcctggcggttgtggttccgaatcctgtgggtgttattgtcgaccgtgcgggtaccgttgtacgcggtaactatgatgctcagaccggtgtgcgtcgccacccggtattcgactaccaggaggcatttgctgaacaggttccgccagtcactgaggccggtaataccaccatttctgccatcgttacaaatgtacgtatgtccccggtcgaactgaatcagttcgctaaacaggttcattcttctatgcaccgtggtatccaaccgtttcacacagatatggacggcgacaccctgtttgctgtaaccactgatgaaatcgatctgccgaccaccccgggttcttctcgcgggcgtctctctgtaaatgctacggctctgggcgcgattgcgagcgaagtaatgtgggacgcagtgctagaggctggcaaa

SEQ ID NO: 11 - protein, 354 aa, synthetic construct
NTTPVHALTDIDGGIAVDPAPRLAGPPVFGGPGNAAFDLAPVRSTGREMLRFDFPGVSIGAAHYEEGPTGATVIHIPAGARTAVDARGGAVGLSGGYDFNHAICLAGGAGYGLEAGAGVSGALLERLEYRTGFAELQLVSSAVIYDFSARSTAVYPDKALGRAALEFAVPGEFPQGRAGAGMSASAGKVDWDRTEITGQGAAFRRLGDVRILAVVVPNPVGVIMDRAGTVVRGNYDAQTGVRRHPVFDYQEAFAEQVPPVTQAGNTTISAIVTNVRMSPVELNQFAKQVHSSMHRGIQPFHTDMDGDTLFAVTTDEIDLPTTPGSSRGRLSVNATALGAIASEVMWDAVLEAGK

SEQ ID NO: 12 - DNA, 1062 bp, synthetic construct
  source 1..1062: mol_type = other DNA
aataccacaccggttcacgcgctgaccgatatcgacggtggtattgcagtcgatccagctccacgtctagctggtcctccagttttcggtggtccgggcaacgctgcttttgacctggcaccggttcgtagcaccggtcgtgaaatgctgcgttttgatttcccgggcgtcagcattggtgccgctcactatgaagaaggtccgaccggtgctaccgtaattcatatcccggcaggcgcccgcactgccgttgacgctcgtggtggtgctgtaggcctgtccggcggctatgattttaaccacgctatttgtctggctggcggcgcaggttatggcctggaagcgggtgcgggtgtaagcggtgctctgctggaacgtctggagtaccgtaccggtttcgcagaattacagctggtgtcttcggctgtgatttacgacttctccgcgcgttccactgccgtttacccggataaagcgctcggccgtgcagcattggaattcgcggtaccgggtgaattcccgcagggtcgtgcgggcgctggcatgtccgcttccgctggtaaggtagactgggatcgtaccgagatcaccggtcagggtgcagctttccgtaggctgggtgacgtgcgtatcctggcggttgtggttccgaatcctgtgggtgttattatggaccgtgcgggtaccgttgtacgcggtaactatgatgctcagaccggtgtgcgtcgccacccggtattcgactaccaggaggcatttgctgaacaggttccgccagtcactcaggccggtaataccaccatttctgccatcgttacaaatgtacgtatgtccccggtcgaactgaatcagttcgctaaacaggttcattcttctatgcaccgtggtatccaaccgtttcacacagatatggacggcgacaccctgtttgctgtaaccactgatgaaatcgatctgccgaccaccccgggttcttctcgcgggcgtctctctgtaaatgctacggctctgggcgcgattgcgagcgaagtaatgtgggacgcagtgctagaggctggcaaa

SEQ ID NO: 13 - protein, 354 aa, Flavobacterium sp.
NTTPVHALTDIDGGIAVDPAPRLAGPPVFGGPGNAAFDLVPVRSTGRETLRFDFPGVSVGSAHYEEGPTGATVIHIPAGARTAVDARGGAVGLSGGYDFNHAICLAGGASYGLEAGAGVSGALLERLEYRTGFAEAQLVSSAVIYDFSARSTAVYPDKALGRAALEFAVPGEFPQGRAGAGMSASAGKVDWDRTEITGQGAAFRRLGDVRILAVVVPNPVGVIMDRAGGIVRGNYDAQTGVRRHPVFDYQEAFAEQLPPVTQAGNTTISAIVTNVRMSPVELNQFAKQVHSSMHRGIQPFHTDMDGDTLFAVTTDEIDLPTTPGSSRGRLSVNATALGAIASEVMWDAVLEAAK

SEQ ID NO: 14 - DNA, 1062 bp, Flavobacterium sp.
  source 1..1062: mol_type = genomic DNA
aataccacaccggtgcatgctctcaccgatatcgacggtggcattgcggtagatccagctccacgtcttgcaggtccacctgtgttcggtggacctggtaacgctgcattcgatctggttccggtacgttccaccggtcgtgaaaccctgcgtttcgattttccgggggtttctgtgggtagcgcgcactacgaagagggtccgactggcgccaccgtcatccacatcccggctggggcacgtactgctgtagatgcacgtggcggcgcagtgggcctgtccggtggctacgacttcaaccacgctatttgtctggctggtggtgcttcttacggcctggaggctggcgcaggcgtttccggtgctctgctggaacgcctcgaataccgcacaggtttcgctgaagcccaactggtgtcttccgcagtaatctacgacttctctgcccgttcaaccgcagtgtatccagacaaagccttgggtcgtgctgcgctggaatttgccgtaccgggtgaattcccacagggtcgcgctggtgccggcatgagcgccagcgcgggcaaagttgattgggatcgtaccgagatcacaggtcagggcgctgctttccgccgtctgggcgatgtgcgtatcctggctgttgttgtgccaaacccagttggcgtaattatggatcgtgcaggcggtatcgtccgtggcaactatgatgcgcaaactggcgttcgccgtcacccagtatttgattaccaggaagctttcgcggaacaactgccgcctgttactcaggcgggcaacacgaccatcagcgcgatcgttaccaatgtccgcatgagtccggtggaactgaatcaattcgccaaacaggtgcatagctctatgcaccgcggtatccagccatttcacaccgatatggacggcgataccctgttcgccgtgactaccgacgaaatcgatctcccgactacgccgggctcctcccgtggccgcctgtctgttaacgcaaccgctctgggcgcaattgcttctgaagttatgtgggacgccgtattagaagcggcgaag

SEQ ID NO: 15 - protein, 354 aa, synthetic construct
NTTPVHALTDIDGGIAVDPAPRLAGPPVFGGPGNAAFDLVPVRSTGRETLRFDFPGVSVGSAHYEEGPTGATVIHIPAGARTAVDARGGAVGLSGGYDFNHAICLAGGAGYGLEAGAGVSGALLERLEYRTGFAELQLVSSAVIYDFSARSTAVYPDKALGRAALEFAVPGEFPQGRAGAGMSASAGKVDWDRTEITGQGAAFRRLGDVRILAVVVPNPVGVIMDRAGGIVRGNYDAQTGVRRHPVFDYQEAFAEQLPPVTQAGNTTISAIVTNVRMSPVELNQFAKQVHSSMHRGIQPFHTDMDGDTLFAVTTDEIDLPTTPGSSRGRLSVNATALGAIASEVMWDAVLEAAK

SEQ ID NO: 16 - DNA, 1062 bp, synthetic construct
  source 1..1062: mol_type = other DNA
aataccacaccggtgcatgctctcaccgatatcgacggtggcattgcggtagatccagctccacgtcttgcaggtccacctgtgttcggtggacctggtaacgctgcattcgatctggttccggtacgttccaccggtcgtgaaaccctgcgtttcgattttccgggggtttctgtgggtagcgcgcactacgaagagggtccgactggcgccaccgtcatccacatcccggctggggcacgtactgctgtagatgcacgtggcggcgcagtgggcctgtccggtggctacgacttcaaccacgctatttgtctggctggtggtgctggttacggcctggaggctggcgcaggcgtttccggtgctctgctggaacgcctcgaataccgcacaggtttcgctgaactccaactggtgtcttccgcagtaatctacgacttctctgcccgttcaaccgcagtgtatccagacaaagccttgggtcgtgctgcgctggaatttgccgtaccgggtgaattcccacagggtcgcgctggtgccggcatgagcgccagcgcgggcaaagttgattgggatcgtaccgagatcacaggtcagggcgctgctttccgccgtctgggcgatgtgcgtatcctggctgttgttgtgccaaacccagttggcgtaattatggatcgtgcaggcggtatcgtccgtggcaactatgatgcgcaaactggcgttcgccgtcacccagtatttgattaccaggaagctttcgcggaacaactgccgcctgttactcaggcgggcaacacgaccatcagcgcgatcgttaccaatgtccgcatgagtccggtggaactgaatcaattcgccaaacaggtgcatagctctatgcaccgcggtatccagccatttcacaccgatatggacggcgataccctgttcgccgtgactaccgacgaaatcgatctcccgactacgccgggctcctcccgtggccgcctgtctgttaacgcaaccgctctgggcgcaattgcttctgaagttatgtgggacgccgtattagaagcggcgaag

(19) United States
(12) Patent Application Publication
(10) Pub. No.: US 2025/0188500 A1
(43) Pub. Date: Jun. 12, 2025

(54) METHODS AND SYSTEMS FOR DEPOLYMERIZING POLYAMIDES

(71) Applicant: Alliance for Sustainable Energy, LLC, Golden, CO (US)

(72) Inventors: Elizabeth Louise BELL, Golden, CO (US); Gregg Tyler BECKHAM, Golden, CO (US)

(21) Appl. No.: 18/974,411

(22) Filed: Dec. 9, 2024

Related U.S. Application Data
(60) Provisional application No. 63/607,947, filed on Dec. 8, 2023.

(51) Int. Cl. C12P 13/00; C08J 11/10; C12N 9/80; C12P 7/44

(52) U.S. Cl. CPC C12P 13/001; C08J 11/105; C12N 9/80; C12P 7/44; C08J 2377/00

(57) ABSTRACT

The present disclosure relates to a method that includes hydrolyzing a polyamide by contacting the polyamide with a hydrolase. In some embodiments of the present disclosure, the polyamide may include nylon-6, nylon 6,6, or a combination thereof. In some embodiments of the present disclosure, the hydrolyzing may produce aminohexanoic acid, an oligomer of aminohexanoic acid, ε-caprolactam, or combinations thereof.

### CROSS-REFERENCE TO RELATED APPLICATIONS

This application claims priority from U.S. Provisional Patent Application No. 63/607,947 filed on Dec. 8, 2023 and its associated appendix, the contents of which are incorporated herein by reference in their entirety.

### CONTRACTUAL ORIGIN

This invention was made with government support under Contract No. De-AC36-08GO28308 awarded by the Department of Energy. The government has certain rights in the invention.

### SEQUENCE LISTING

The instant application contains a Sequence Listing which has been submitted electronically and is hereby incorporated by reference in its entirety. The XML copy as filed herewith was originally created on Feb. 11, 2025 is named NREL 23-106.xml and is 26 kilobytes in size.

### BACKGROUND

Polyamides (PAs), often referred to as nylons, are versatile, amide bond-linked, petroleum-derived thermoplastics. Considered high-performance materials, PAs are used in a range of applications, including in textiles, fishing nets, packaging, and medical devices. The most prevalent nylons are PA6 and PA6,6, derived from caprolactam or adipic acid and hexamethylene diamine monomers, respectively, with a combined annual global consumption of 7.67 million tons in 2018. The production of PA6 and PA6,6 is both energy- and greenhouse gas (GHG) emissions-intensive, contributing a combined 197 MJ/kg of energy use and 10.4 kg-CO₂e/kg GHG emissions for US nylon consumption alone. Furthermore, as with many anthropogenic polymers, PA6 and PA6,6 are sparingly biodegradable, hence they accumulate in the environment. Circularization of the nylon economy via recycling offers a potential avenue for reducing energy consumption and GHG emissions, decreasing reliance upon fossil fuels, and minimizing environmental impacts of PA waste. However, little postconsumer nylon waste is currently collected, and recycling proves challenging as PAs are often combined with other polymers. Hence, there remains a need for a wider catalog of nylon recycling approaches to manage this polymer at end-of-life, with selective techniques that release PA monomers being of particular interest for closed-loop recycling.

### SUMMARY

An aspect of the present disclosure is a method that includes hydrolyzing a polyamide by contacting the polyamide with a hydrolase. In some embodiments of the present disclosure, the polyamide may include nylon-6, nylon 6,6, or a combination thereof. In some embodiments of the present disclosure, the hydrolyzing may produce 6-aminohexanoic acid, an oligomer of 6-aminohexanoic acid, ε-caprolactam, or combinations thereof.

In some embodiments of the present disclosure, the oligomer may be a compound of at least one of Structure (I) or Structure (II):

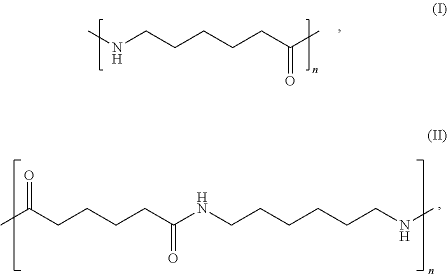

wherein n is between 2 and 5.

In some embodiments of the present disclosure, the hydrolase may be selected from an SHD-hydrolase, an NylB-type hydrolase, an NylC-type hydrolase, and combinations thereof. In some embodiments of the present disclosure, the hydrolase may include an amino acid sequence that is at least 90% identical to SEQ ID NO: 1. In some embodiments of the present disclosure, the hydrolase may include an amino acid sequence according to SEQ ID NO: 1 having an R-S mutation at residue 186, an F-C mutation at residue 263, a D-Y mutation at residue 369, or any combination thereof. In some embodiments of the present disclosure, the hydrolase may be encoded by a nucleic acid sequence that is at least 90% identical to SEQ ID NO: 2.

In some embodiments of the present disclosure, the hydrolase may include an amino acid sequence that is at least 90% identical to SEQ ID NO: 5. In some embodiments of the present disclosure, the hydrolase may nclude an amino acid sequence according to SEQ ID NO: 5 having an S-G mutation at residue 110, an A-L mutation at residue 136, an E-Q mutation at residue 262, or any combination thereof. In some embodiments of the present disclosure, the hydrolase may be encoded by a nucleic acid sequence that is at least 90% identical to SEQ ID NO: 6.

In some embodiments of the present disclosure, the hydrolase may include an amino acid sequence that is at least 90% identical to SEQ ID NO: 9. In some embodiments of the present disclosure, the hydrolase may include an amino acid sequence according to SEQ ID NO: 9 having a D-A mutation at residue 35, a D-G mutation at residue 121, an H-Y mutation at residue 129, a V-M mutation at residue 224, an E-Q mutation at residue 262, or any combination thereof. In some embodiments of the present disclosure, the hydrolase may be encoded by a nucleic acid sequence that is at least 90% identical to SEQ ID NO: 10.

In some embodiments of the present disclosure, the hydrolase may include an amino acid sequence that is at least 90% identical to SEQ ID NO: 13. In some embodiments of the present disclosure, hydrolase may include an amino acid sequence according to SEQ ID NO: 13 having a S-G mutation at residue 110, a A-L mutation at residue 136, or any combination thereof. In some embodiments of the present disclosure, the hydrolase may be encoded by a nucleic acid sequence that is at least 90% identical to SEQ ID NO: 14.

In some embodiments of the present disclosure, the contacting may be performed in an aqueous solution. In some embodiments of the present disclosure, the contacting may be performed at a temperature between 22° C. and 100° C. In some embodiments of the present disclosure, the aqueous solution may further include bovine serum albumin (BSA).

### BRIEF DESCRIPTION OF DRAWINGS

Some embodiments are illustrated in referenced figures of the drawings. It is intended that the embodiments and figures disclosed herein are to be considered illustrative rather than limiting.

FIG. 1 illustrates that nylonase activity is found across enzymes with different active site architectures and diverse substrate preferences. Panel A illustrates example catalytic residues used by potential nylonases: Ser-His-Asp (SHA) serine hydrolases, NylB-type serine hydrolases (putative catalytic triad for this enzyme group, only two known enzymes described), amidases (example amidase catalytic triad used by Ser-(cis)Ser-Lys amidases), and NylC-type N-terminal nucleophile hydrolases. Panel B illustrates a phylogenetic tree of enzymes selected for potential PA6 deconstruction activity. Inset numbers on the branches represent confidence values (%).

FIG. 2 summarizes the 41 nylonases evaluated in the work described herein.

FIG. 3 illustrates melt curves for thermostabilized NylC variants. Melt curve readings were carried out in triplicate.

FIG. 4 illustrates N-butyl-4-nitroaniline (NB4N) activity assays with potential nylonases. All successfully expressed enzymes were tested for their ability to cleave the amide bond in NB4N, leading to the release of p-nitroaniline (p-NA), which can be monitored spectrophotometrically. The bar chart shows the conversion extent of NB4N to p-NA after 5 h of reaction using 0.5 mM NB4N and 2 μM enzyme at 30° C. in reaction buffer (100 mM sodium phosphate buffer (NaPi), pH 7.5, 150 mM NaCl). Proteases exhibited no detectable activity and thus are not shown. A double asterisk (**) indicates reactions with enzymes that went to completion in ≤5 mins, a single asterisk (*) indicates reactions with enzymes which went to completion in ≤30 mins. All reactions are ordered with respect to reaction rate, with the fastest enzymes to the left. Reactions were carried out in triplicate; error bars show the standard deviation of the replicate measurements; each replicate measurement is represented as a grey circle.

FIG. 5 illustrates activity assays with N-(4-nitrophenyl) butanamide (N4NB) for all enzymes. p-NA (p-nitroaniline) release profiles of all enzymes tested. Reactions contained 0.5 mM N4NB substrate and 2 μM enzyme in 100 mM sodium phosphate buffer (NaPi), pH 7.5, 150 mM NaCl, and were monitored over the course of 24 h at 30° C. Enzymes are plotted by grouping: Panel A—NylB-type, Panels B & C—NylC-type, Panels D, E & F—Amidases and Misc., Panel G—Proteases, and Panels H & I—SHD-hydrolases. Points represent the mean of triplicate measurements, with the error bars representing the standard deviations of replicate measurements being too small to be visualized. Note that y-axes are shown on different scales to enable different levels of activity to be seen.

FIG. 6 illustrates LC-MS/MS analysis of major soluble compounds present in enzymatic PA6 deconstruction reactions. Panel A illustrates a representative LC-MS/MS chromatogram following enzymatic hydrolysis of PA6, showing the eight compounds that were detected and quantified. Panel B illustrates example calibration plots for the LC-MS/MS analysis method, showing the five chemical standards (analytes) that were available commercially, measured from 0.01-7.0 μg mL⁻¹. The 6-AHA tetramer and pentamer concentrations, and the 6-AHA cyclic trimer concentrations were determined using the calibration curves from the 6-AHA trimer and 6-AHA cyclic dimer, respectively, as no commercial standards were available.

FIG. 7 illustrates materials characterization before and after enzymatic PA6 film deconstruction reactions. Summary of DSC, GPC and TGA data collected throughout the study. The polydispersity index is represented by Ð. For the sample preparation, “unmodified” samples were analyzed as received from the supplier or as originally synthesized, apart from DSC measurements, where the PA6 was dried at 40° C. for 24 h prior to analysis to remove any residual water, “washed” samples were analyzed following the washing protocol described in the Methods prior to reaction, “no-enzyme” samples were incubated in reaction buffer (100 mM NaPi buffer, 150 mM NaCl, pH 7.5) for the described amount of time at the stated temperature in the absence of enzyme, and “+NylCK-TS” samples were analyzed following enzymatic reactions for the described amount of time at the stated temperature (3.25 μM NylCK-TS/mg PA6, 0.65% wt PA6 substrate loading, 100 mM pH 7.5 NaPi buffer with 150 mM NaCl). Dashes represent where no data was collected for that condition set. The mass loss measured during TGA analysis can be attributed to water loss as it occurred at around 100° C. * All Goodfellow PA6 film used in these analyses came from one batch, as there were small differences between samples of material received from Goodfellow

FIG. 8 illustrates materials characterization plots for Goodfellow PA6 film. The plots are from untreated PA6 film (0.2 mm thickness) as received from Goodfellow (unmodified). Panel A illustrates a differential scanning calorimetry (DSC) plot. For DSC, the PA6 was dried at 40° C. for 24 h prior to analysis to remove any residual water. Panel B illustrates a gel permeation chromatography (GPC) plot measured with the differential refractive index (dRI) detector. Panel C illustrates a thermogravimetric (TGA) plot. For the TGA plot, the darker lines represent the weight (%) (left y-axis), and the lighter lines the derivative weight (%) (right y-axis). Plots represent single measurements.

FIG. 9 a reaction profile of enzymatic PA6 deconstruction. Total released linear 6-AHA oligomers, represented as 6-AHA monomer equivalents, in reactions with 2 μM NylC_{K}-TS (6.5 μM enzyme/mg PA6) and 13 mg PA6 (0.65 wt % substrate loading) at 60° C. over the course of 14 days, in reaction buffer (100 mM NaPi buffer, pH 7.5, 150 mM NaCl). Reactions were carried out in triplicate; error bars represent the standard deviation of the replicate measurements; circles represent the mean value of the triplicate measurements.

FIG. 10 illustrates the release of PA6 oligomers during reaction. Panel A illustrates that there was minimal release of linear-oligomers of 6-AHA following incubation of PA6 film (13 mg) in reaction buffer (100 mM NaPi, pH 7.5, 150 mM NaCl) over the course of 10 days from 40-70° C. Panel B illustrates the total released cyclic 6-AHA oligomers following reaction with enzyme (2 μM LCC-ICCG, 6.5 μM enzyme/mg PA6) and 13 mg PA6 (0.65 wt % substrate loading) or without enzyme over the course of 10 days (60° C., 100 mM NaPi buffer, pH 7.5, 150 mM NaCl). Cyclic oligomer concentrations shown here are representative of all enzymes apart from NylC-type and GatA. For both charts, reactions were carried out in triplicate, error bars show the standard deviation of the replicate measurements, and the replicate measurements are represented as circles.

FIG. 11 illustrates enzymatic PA6 depolymerization reactions with the top performing enzymes. Panel A illustrates heat map time-course profiles of the total released (sum) 6-AHA monomer equivalents following PA6 depolymerization reactions with the top performing SHA-hydrolases (pink), amidases (purple), NylB-type (blue), and NylC-type (yellow) enzymes carried out from 40-70° C. over 10 days. The heat map gradient represents the total released 6-AHA monomer equivalents from 0-360 μM, with each square representing the average of reactions carried out in triplicate. Panel B illustrates representative depolymerization reactions with enzymes from each group at their optimal temperatures (x-axis), with 6-AHA oligomers of different lengths represented as their 6-AHA monomer equivalents. Reactions were carried out in triplicate, error bars are the standard deviation of the replicate measurements, and each replicate measurement is represented as a grey circle. For all data in panel A and B, the reactions contained 2 μM enzyme (6.5 μM enzyme/mg PA6), 13 mg PA6 (0.65 wt % substrate loading) and were incubated in reaction buffer (100 mM sodium phosphate buffer (NaPi), pH 7.5, 150 mM NaCl).

FIG. 12 illustrates reactions of NylC-type enzymes with PA6. Total released linear 6-AHA oligomers from enzymes in the NylC-type group, following reaction with PA6 film. Reactions contained 2 μM enzyme (6.5 μM enzyme/mg PA6), 13 mg PA6 (0.65 wt % substrate loading), and were incubated from 40-70° C. over the course of 10 days in reaction buffer (100 mM NaPi buffer, pH 7.5, 150 mM NaCl). Above 50° C., reactions with enzymes that led to no detectable product release above background are not shown. Reactions were carried out in triplicate, error bars show the standard deviation of the replicate measurements, and the replicate measurements are represented as circles.

FIG. 13 illustrates reactions of NylB-type enzymes with PA6. Total released linear 6-AHA oligomers from enzymes in the NylB-type group, following reaction with PA6 film. Reactions contained 2 μM enzyme (6.5 μM enzyme/mg PA6), 13 mg PA6 (0.65 wt % substrate loading) and were incubated from 40-70° C. over the course of 10 days in reaction buffer (100 mM NaPi buffer, pH 7.5, 150 mM NaCl). Above 50° C., minimal product release above background was seen with any of the NylB-type enzymes. Reactions were carried out in triplicate, error bars show the standard deviation of the replicate measurements, and the replicate measurements are represented as circles.

FIG. 14 illustrates reactions of SHD-hydrolases with PA6. Total released linear 6-AHA oligomers with enzymes in the SHD-hydrolase group following reaction with PA6 film. Reactions contained 2 μM enzyme (6.5 μM enzyme/mg PA6), 13 mg PA6 (0.65 wt % substrate loading) and were incubated from 40-70° C. over the course of 10 days in reaction buffer (100 mM NaPi buffer, pH 7.5, 150 mM NaCl). Above 50° C., reactions with enzymes that led to no detectable product release above background are not shown. Reactions were carried out in triplicate, error bars show the standard deviation of the replicate measurements, and the replicate measurements are represented as circles.

FIG. 15 illustrates reactions of amidases with PA6. Total released linear 6-AHA oligomers with enzymes in the amidase group following reaction with PA6 film. Reactions contained 2 μM enzyme (6.5 μM enzyme/mg PA6), 13 mg PA6 (0.65 wt % substrate loading) and were incubated from 40-70° C. over the course of 10 days in reaction buffer (100 mM NaPi buffer, pH 7.5, 150 mM NaCl). Above 50° C., reactions with enzymes that led to no detectable product release above background are not shown. Reactions were carried out in triplicate, error bars show the standard deviation of the replicate measurements, and the replicate measurements are represented as circles.

FIG. 16 illustrates reactions of proteases, AfNitB and RoL10X with PA6. Total released linear 6-AHA oligomers from enzymes not in one of the four main groups (misc.), following reaction with PA6 film. Reactions contained 2 μM enzyme (6.5 μM enzyme/mg PA6), 13 mg PA6 (0.65 wt % substrate loading), and were incubated from 40-70° C. over the course of 10 days in reaction buffer (100 mM NaPi buffer, pH 7.5, 150 mM NaCl). No product release above background levels was seen at any reaction condition. Reactions were carried out in triplicate, error bars show the standard deviation of the replicate measurements, and the replicate measurements are represented as grey.

FIG. 17 illustrates the concentration of 6-AHA cyclic-trimer in reactions with the most active NylCs. Concentrations of 6-AHA cyclic-trimer following reactions of NylC-type enzymes with PA6 film versus a no-enzyme control. For enzymatic reactions, reactions contained 2 μM enzyme (6.5 μM enzyme/mg PA6), 13 mg PA6 (0.65 wt % substrate loading) and were incubated at the enzyme's optimal temperature (x-axis) over the course of 10 days in reaction buffer (100 mM NaPi buffer, pH 7.5, 150 mM NaCl). No-enzyme reactions were treated in the same way, but without enzyme addition. Reactions were carried out in triplicate, error bars show the standard deviation of the replicate measurements, and the replicate measurements are represented as circles.

FIG. 18 illustrates reactions of enzymes with 6-AHA linear oligomers. Panel A illustrates the reaction of NylC_{K}-TS with 6-AHA linear trimer for 24 h at reaction conditions (50 μM 6-AHA trimer, pH 7.5 NaPi buffer, 150 mM NaCl, 2 μM enzyme, 60° C.), showing the change in 6-AHA oligomers as a percentage of total oligomers in the reaction. Panel B illustrates the reaction of Tt-NylC with 6-AHA linear trimer for 24 h at reaction conditions (100 μM 6-AHA trimer, pH 7.5 NaPi buffer, 150 mM NaCl, 2 μM enzyme, 60° C.), showing the change in 6-AHA oligomers as a percentage of total oligomers in the reaction. Panel C illustrates the reaction of NylC_{K}-TS with 6-AHA linear dimer for 24 h at reaction conditions (50 μM 6-AHA dimer, pH 7.5 NaPi buffer, 150 mM NaCl, 2 μM enzyme, 60° C.), showing the change in 6-AHA oligomers as a percentage of total oligomers in the reaction. For both 6-AHA dimer and 6-AHA trimer, there was no change in concentration in no-enzyme control reactions. For Panels A, B, and C, reactions were carried out in triplicate; error bars represent the standard deviation of the replicate measurements; each replicate measurement is represented as circles.

FIG. 19 illustrates reactions of enzymes with 6-AHA linear oligomers. Panel A illustrates the reaction of NylB′-SCY with 6-AHA linear dimer for 24 h at reaction conditions (50 μM 6-AHA dimer, pH 7.5 NaPi buffer, 150 mM NaCl, 2 μM enzyme, 70° C.), showing the change in 6-AHA oligomers as a percentage of total oligomers in the reaction. Panel B illustrates the reaction of NylB′-SCY with 6-AHA linear trimer for 24 h at reaction conditions (50 μM 6-AHA trimer, pH 7.5 NaPi buffer, 150 mM NaCl, 2 μM enzyme, 70° C.), showing the change in 6-AHA oligomers as a percentage of total oligomers in the reaction. Panel C illustrates the reaction of LCC-ICCG with 6-AHA linear trimer for 24 h at reaction conditions. (50 μM 6-AHA trimer, pH 7.5 NaPi buffer, 150 mM NaCl, 2 μM enzyme, 70° C.), showing the change in 6-AHA oligomers as a percentage of total oligomers in the reaction. Panel D illustrates the reaction of GatA with 6-AHA linear dimer for 24 h at reaction conditions (100 μM 6-AHA dimer, pH 7.5 NaPi buffer, 150 mM NaCl, 2 μM enzyme, 70° C.), showing the change in 6-AHA oligomers as a percentage of total oligomers in the reaction. For both 6-AHA dimer and 6-AHA trimer, there was no change in concentration in no-enzyme control reactions. For A, B, C and D, reactions were carried out in triplicate; error bars represent the standard deviation of the replicate measurements.

FIG. 20 illustrates the AlphaFold predicted structure of NfPolyA. The green ribbon represents the structure of NfPolyA as predicted using AlphaFold. The catalytic triad is shown in the center of the protein, in stick representation colored by all atoms, with pink carbon atoms

FIG. 21 illustrates the concentration of 6-AHA cyclic-oligomers in reactions with GatA. Panel A illustrates the concentration of 6-AHA cyclic-dimer, following reaction of GatA with PA6 film versus a no-enzyme control. Panel B illustrates the concentration of 6-AHA cyclic-trimer, following reaction of GatA with PA6 film, versus a no-enzyme control. For enzymatic reactions shown in A and B, reactions contained 2 μM enzyme (6.5 μM enzyme/mg PA6), 13 mg PA6 (0.65 wt % substrate loading), and were incubated from 40° C. over the course of 10 days in reaction buffer (100 mM NaPi buffer, pH 7.5, 150 mM NaCl). No-enzyme reactions were treated in the same way, but without enzyme addition. Reactions were carried out in triplicate, error bars show the standard deviation of the replicate measurements, and the replicate measurements are represented as circles

FIG. 22 illustrates the exploration of reaction conditions on enzymatic PA6 depolymerization. Panel A illustrates the total released linear 6-AHA oligomers, represented as 6-AHA monomer equivalents, in reactions with 1 μM NylC_{K}-TS (60° C.), Tt-NylC (60° C.), or NylB′-SCY (50° C.), over the course of 10 days, varying the substrate loading from 1-5 PA6 squares (6.5-32.5 mg PA6, substrate loading of 0.32-1.6 wt %). Pie-charts represent the proportion of different 6-AHA oligomers measured at that time point. Reactions were carried out in triplicate, error bars are the standard deviation of the replicate measurements, and circle, square, and triangle points represent the mean value of the triplicate measurements. Panel B illustrates PA6 depolymerization reactions with varying enzyme loadings of NylC_{K}-TS (60° C.), Tt-NylC (60° C.), or NylB′-SCY (50° C.). Reactions contained 13 mg PA6 substrate (0.65 wt % substrate loading) and were monitored for 10 days. Reactions were carried out in duplicate, and each replicate measurement is represented as a circle. For all data in Panel A and B, the reaction buffer consisted of 100 mM sodium phosphate buffer (NaPi), pH 7.5, 150 mM NaCl.

FIG. 23 illustrates the total released linear 6-AHA oligomers, represented as 6-AHA monomer equivalents, in reactions with 1 μM NylC_{K}-TS (60° C.), Tt-NylC (60° C.) or NylB′-SCY (50° C.) (3.2 μM enzyme/mg PA6), 13 mg PA6 (0.65 wt % substrate loading), over the course of 10 days, in reaction buffers from pH 6-10 (100 mM buffer, pH 6: citrate, pH7/8: NaPi, pH 9/10 Gly-OH, 150 mM NaCl) at 60° C. Reactions were carried out in triplicate; error bars represent the standard deviation of the replicate measurements; circle, square, and triangle points represent the mean value of the triplicate measurements.

FIG. 24 illustrates crystallinity changes in no-enzyme control reactions over time at different temperatures. Crystallinity changes of PA6 film were determined by DSC over the course of 10 days. For each temperature from 40-70° C., 13 mg PA6 film (0.65 wt % substrate loading) was incubated in reaction buffer (100 mM NaPi buffer, pH 7.5, 150 mM NaCl). Points represent single measurements.

FIG. 25 illustrates reaction profiles of NylC_{K}-TS PA6 deconstruction to understand reaction plateau. f A illustrates the total released linear 6-AHA oligomers, represented as 6-AHA monomer equivalents, in reactions with 1 μM NylC_{K}-TS, (3.2 μM enzyme/mg PA6), 13 mg PA6 (0.65 wt %), over the course of 10 days. PA6 film squares were incubated in reaction buffer for either 0 days (Normal reaction), 3 days or 7 days prior to enzyme addition. Panel B illustrates the total released linear 6-AHA oligomers in reactions with 1 μM NylC_{K}-TS, (3.2 μM enzyme/mg PA6), 13 mg PA6 (0.65 wt % substrate loading) over the course of 10 days, supplemented with 2 μM bovine serum albumin (BSA). Panel C illustrates the total released linear 6-AHA oligomers in reactions with 0.01 μM NylC_{K}-TS (0.032 μM enzyme/mg PA6), 13 mg PA6 (0.65 wt % substrate loading) over the course of 10 days, supplemented with 0.5-2.0 μM BSA. Panel D illustrates the total released linear 6-AHA oligomers in reactions started with 1 μM NylC_{K}-TS, (3.2 μM enzyme/mg PA6), 13 mg PA6 (0.65 wt % substrate loading) (normal reaction), where following reaction for 7 days, either fresh enzyme (1 μM) or fresh PA6 film (13 mg) was added, and the reaction allowed to progress. For A, B, C and D, reactions were conducted in 100 mM NaPi buffer, pH 7.5, 150 mM NaCl at 60° C.; reactions were carried out in triplicate; error bars represent the standard deviation of the replicate measurements; circle, square and triangle points represent the mean value of the triplicate measurements.

FIG. 26 illustrates the exploration of PA depolymerization plateau using NylC_{K}-TS as a test case. Panel A illustrates the total released linear 6-AHA oligomers following PA6 film depolymerization reactions (13 mg PA6, 0.65 wt % substrate loading), using either 0.01 μM or 1 μM NylC_{K}-TS enzyme alone (0.032 μM enzyme mg/PA6 or 3.2 μM enzyme mg/PA6, respectively) compared to a reaction containing 0.01 μM NylC_{K}-TS supplemented with 0.5 μM bovine serum albumin (BSA) over the course of 10 days. Panel B illustrates the total released linear 6-AHA oligomers in reactions with 1 μM NylC_{K}-TS, (3.2 μM enzyme/mg PA6) and 13 mg PA6 (0.65 wt % substrate loading), where the reaction was either allowed to progress for 13 days with no interruption (Normal reaction), where fresh enzyme was added to the reaction after three days (1 μM NylC_{K}-TS, +Fresh enzyme), or where fresh substrate was added to the reaction after 3 days (13 mg PA6, +Fresh substrate), and the reactions monitored for a further 10 days. For Panels A and B, circle, square, and triangle points represent the mean value of the triplicate measurements. Panel C illustrates PA6 depolymerization reactions with 1 μM NylC_{K}-TS, (3.2 μM enzyme/mg PA6), and 13 mg PA6 (0.65 wt % substrate loading), with either low crystallinity Goodfellow film (13% crystallinity), or high crystallinity PA6 film (23% crystallinity). Replicate measurements are represented as grey circles. For Panels A-C, reactions were carried out in triplicate, and error bars represent the standard deviation of the replicate measurements. Panel D illustrates SEM images of PA6 films either without enzyme or with 1 μM NylC_{K}-TS, (3.2 μM enzyme/mg PA6, 13 mg PA6 (0.65 wt % substrate loading)). Ten images were collected for each experiment, with those shown being representative of the set. Scale bar, 100 μm. For panels A-D, all reactions were carried out in reaction buffer 100 mM sodium phosphate buffer (NaPi), pH 7.5, 150 mM NaCl at 60° C.

FIG. 27 illustrates substrate characterization of PA6 films incubated with and without enzyme. Panel A illustrates a comparison of GPC plots, measured with the differential refractive index (dRI) detector. Panel B illustrates DSC plots and Panel C TGA plots for reactions with or without enzyme after 10 days of incubation at 60° C. versus the washed PA6 film prior to reaction. No-enzyme reactions contained 13 mg PA6 (0.65 wt % substrate loading) in reaction buffer (100 mM NaPi buffer, pH 7.5, 150 mM NaCl). NylC_{K}-TS reactions contained 1 μM NylC_{K}-TS (3.2 μM enzyme/mg PA6), and 13 mg PA6 (0.65 wt %) in reaction buffer (100 mM NaPi buffer, pH 7.5, 150 mM NaCl). For the TGA plots, the darker colored lines represent the weight (%) (left y-axis), and the lighter lines the derivative weight (%) (right y-axis). Plots represent single measurements.

FIG. 28 illustrates DSC traces for a Goodfellow PA6 film and a crystalline PA6 film. DSC plots of the in-house prepared crystalline PA6 film (23.0% crystallinity) versus the Goodfellow PA6 film (13.2% crystallinity) used in enzyme reactions. For DSC, the PA6 was dried at 40° C. for 24 h prior to analysis to remove any residual water. The DSC analysis presented here was conducted on the unmodified samples prior to washing or reaction. Plots represent single measurements

FIG. 29 illustrates SEM images of PA6 films incubated with NylC_{K}-TS. Example SEM images of PA6 films incubated in reaction buffer (100 mM NaPi buffer, pH 7.5, 150 mM NaCl) at 60° C. for 10 days, either without enzyme (no enzyme) or with 1 μM NylC_{K}-TS (3.2 μM enzyme/mg PA6), and 13 mg PA6 (0.65 wt % substrate loading). SEM images show additional interesting features of the PA6 film following NylC_{K}-TS depolymerizations. Ten images were collected for each experiment, with those shown being representative of the set. Scale bar either 10 or 100 μm as detailed in the image.

FIGS. 30-33 illustrate comparisons of enzyme point mutations in thermostabilized strains relative to wildtype strains. FIG. 30 illustrates a comparison of NylB′ (aa) (SEQ ID NO: 1) to NylB′-SCY (aa) (SEQ ID NO: 3). FIG. 31 illustrates a comparison of NylC_{A} (aa) (SEQ ID NO: 5) to NylC_{A}-TS (aa) (SEQ ID NO: 7). FIG. 32 illustrates a comparison of NylCₚ₂ (aa) (SEQ ID NO: 9) to NylCₚ₂-TS (aa) (SEQ ID NO: 11). FIG. 33 illustrates a comparison of NylC_{K} (aa) (SEQ ID NO: 13) to NylC_{K}-TS (aa) (SEQ ID NO: 15).

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

The embodiments described herein should not necessarily be construed as limited to addressing any of the particular problems or deficiencies discussed herein. References in the specification to “one embodiment”, “an embodiment”, “an example embodiment”, “some embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The present disclosure relates to enzymes that can depolymerize polyamides, e.g., nylon-6 (PA6) and nylon-6,6 (PA6,6), in addition to polyamide pre-treatment steps that can lead to improved nylon depolymerization, for example solvent contacting and particle size reduction that can, among other things, improve the accessibility of the enzymes to a larger percentage of the polyamide substrates.

Specifically, the present disclosure describes a study of over 30 potential natural and engineered nylon-hydrolyzing enzymes (nylonases), using mass spectrometry to quantify eight compounds resulting from enzymatic PA6 hydrolysis. Time-course reactions from 40-70° C. showcase enzyme-dependent variations in product distributions and extent of PA6 (in the form of a film) depolymerization. The most active nylonase identified and described herein is a thermostabilized variant of NylC_{K}, a hydrolase.

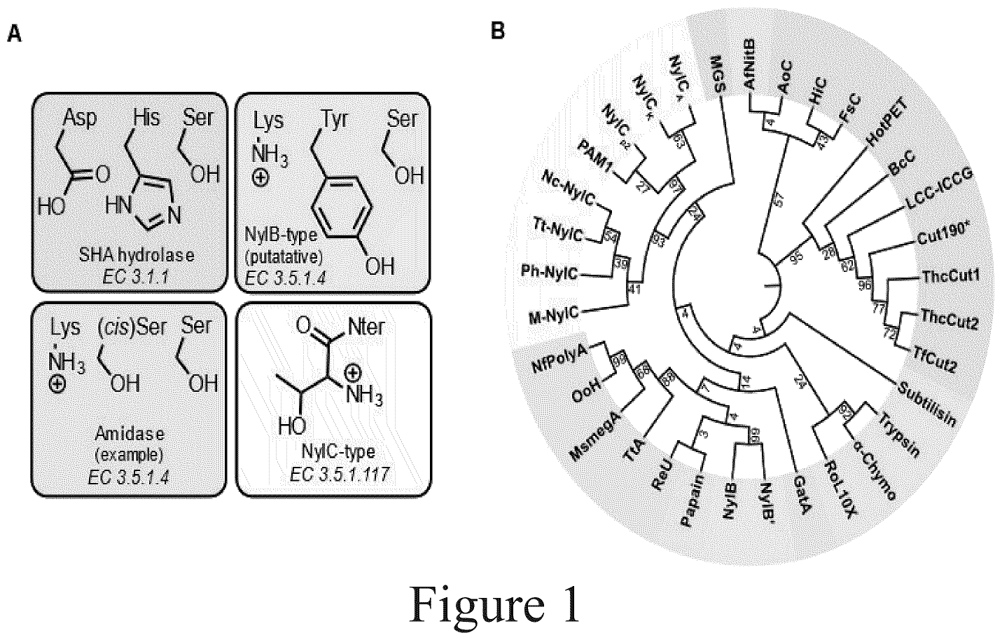

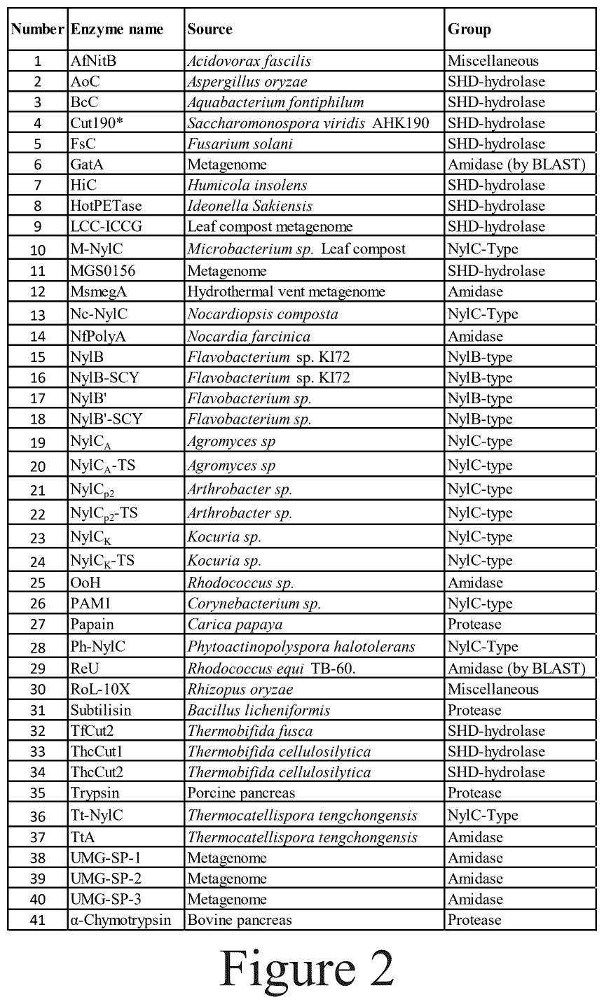

41 nylonases were evaluated for their potential for PA6 depolymerization (sec Panel B of FIG. 1), selecting those with a range of enzyme active-site architectures and a range of substrate preferences. The enzymes evaluated are summarized in FIG. 2. This set of enzymes included engineered enzymes with high thermostability, a desirable characteristic for polymer deconstruction. These enzymes were tested for their ability to hydrolyze PA6 films, using a high-throughput nylon degradation analysis method based on liquid chromatography with tandem mass spectrometry (LC-MS/MS), which accurately detects linear monomers of PA6 up to pentamers, and cyclic oligomers up to trimers. The most promising enzyme candidates were subjected to additional characterization to explore depolymerization extent as a function of reaction conditions. Because PA6 hydrolysis plateaued in all cases, to better understand the depolymerization process, the reaction progression of the top-performing enzyme was studied, a thermostabilized NylC homologue. The results of these studies indicate that although measurable nylonase activity was present in multiple enzymes, with different enzyme-dependent distributions of reaction products, Ntn hydrolases appeared to be the most active.

Identification, expression, and small molecule amide bond hydrolysis assays of potential nylonases: As polymer chains become more accessible near the glass transition temperature of the substrate (for PA6, T_{g} ˜50° C.), enzyme thermotolerance is a desirable characteristic. Ntn hydrolases and AS family enzyme candidates were selected following NylCₚ₂ and NfPolyA homologue searches, selecting enzymes from potentially thermotolerant sources. Following selection, enzymes were broadly categorized into six main groupings dependent on both their catalytic activities and previously described substrate preferences (see Panel B of FIG. 1). The groupings used from here on are as follows: (1) NylC-type enzymes, Ntn-hydrolases found to hydrolyze PA6 trimers and longer PA oligomers, (2) NylB-type enzymes, serine hydrolases (Ser-Tyr-Lys catalytic triad) found to specifically hydrolyze PA dimers, (3) amidases, AS family enzymes that act on linear amides, (4) Ser-His-Asp hydrolases (SHA-hydrolases), serine hydrolases with a substrate preference for ester-linked polymers, (5) proteases, serine proteases that cleave peptide bonds in proteins, and (6) miscellaneous (misc.), enzymes that do not fall into one of the other five main groupings.

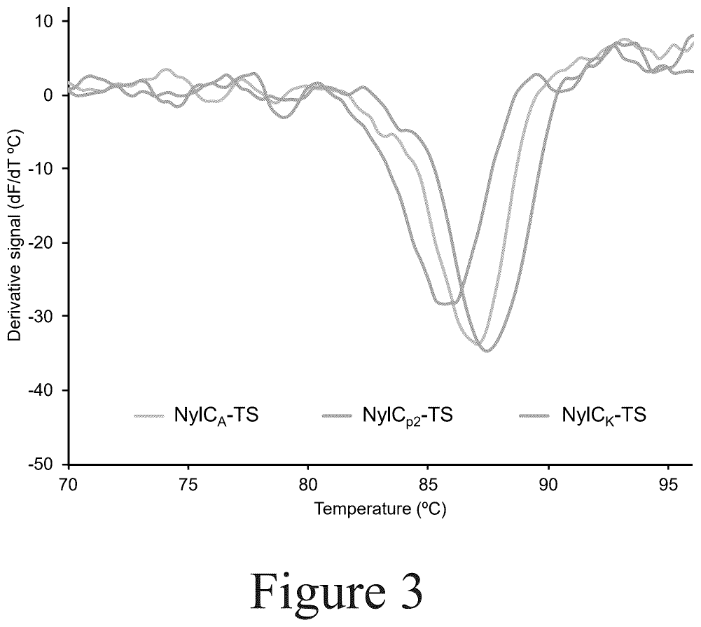

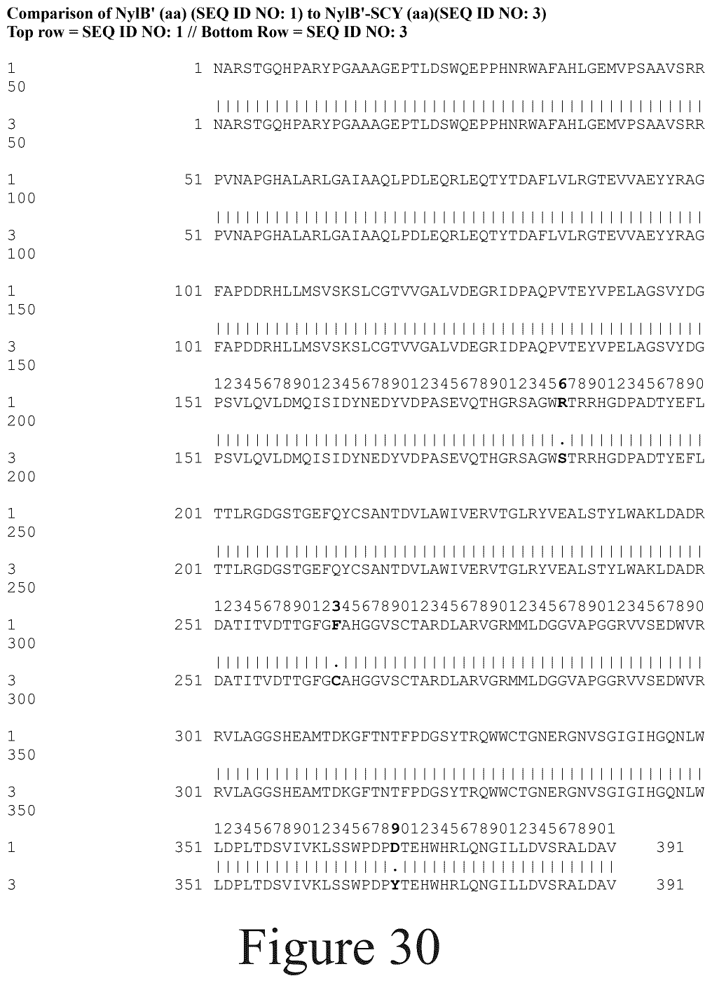

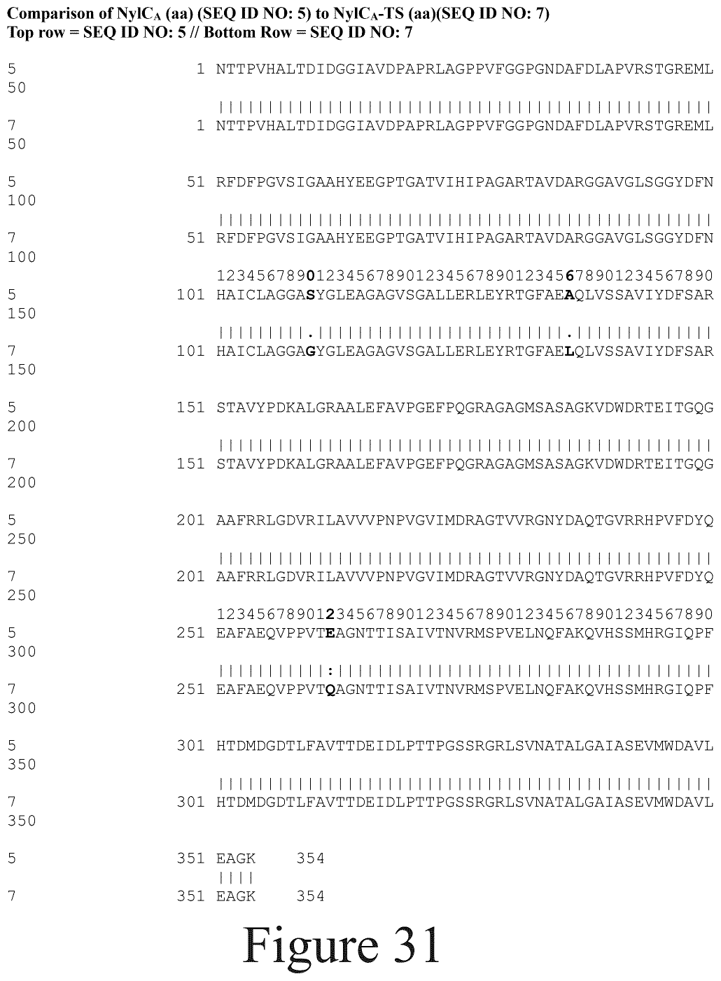

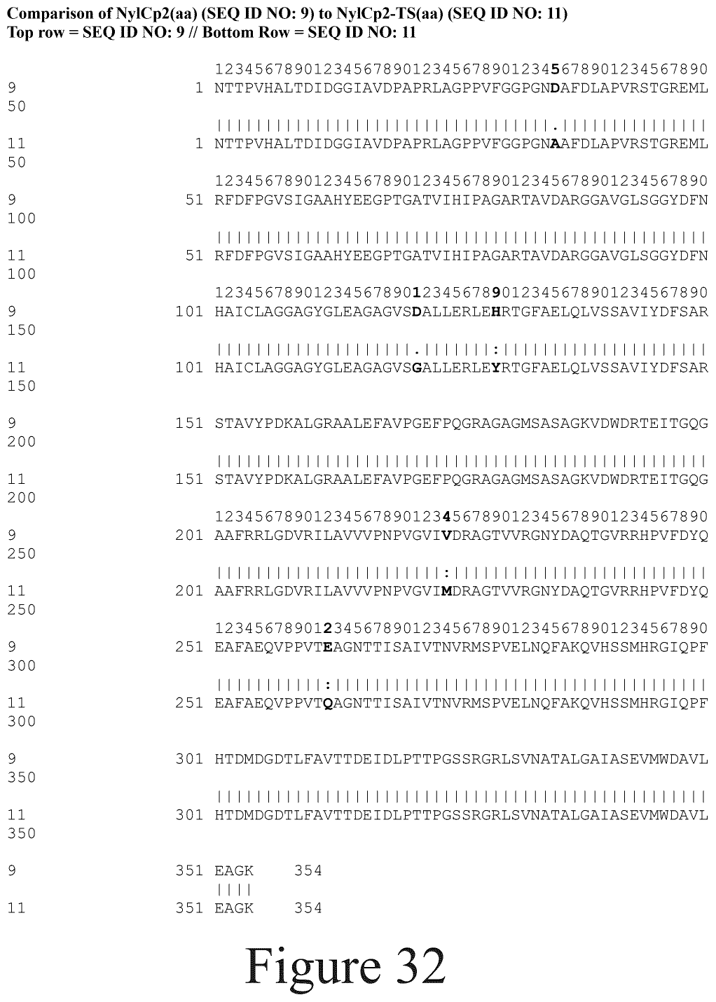

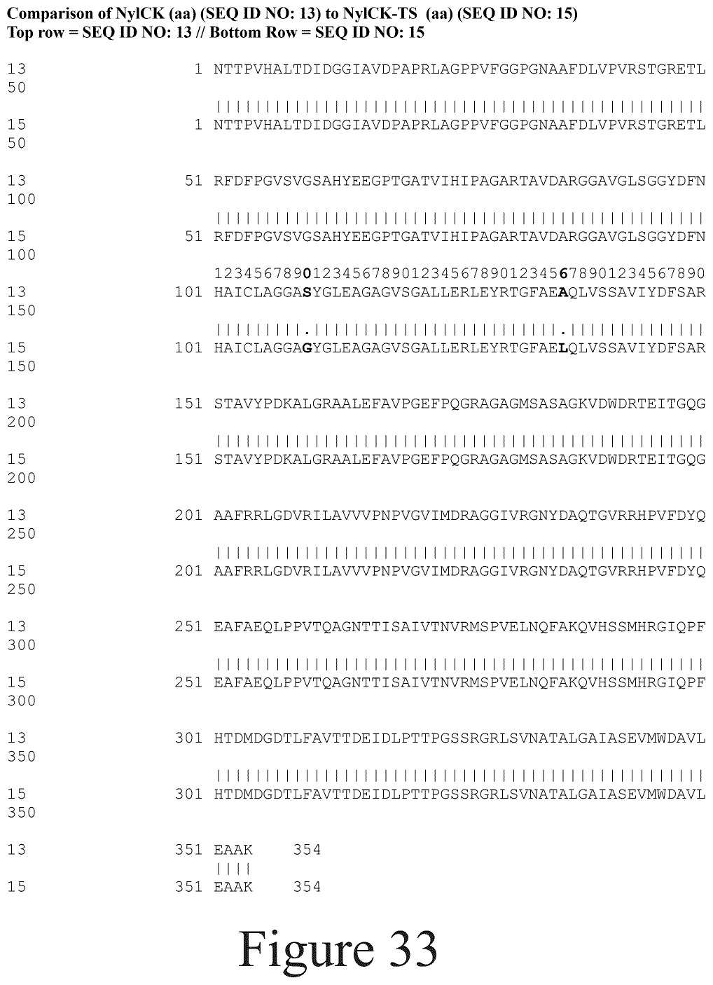

NylCₚ₂ was thermostabilized by D122G/H130Y/D36A/E263Q, with a Tₘ increase of 36° C., demonstrating hydrolytic activity on solid PA6 powder. NylCs was rationally mutated from Agromyces (NylC_{A}) and Kocuria (NylC_{K}) to increase their melting temperatures (Tₘ). In both enzymes, residues previously described as potentially destabilizing were substituted for their homologous counterparts in NylCₚ₂ (S111G and A137L), with E263Q additionally being introduced into NylC_{A} as a potential stabilizing mutation from NylC_{K}. These mutations increased protein Tₘ by 16.4° C. and 25.1° C. for NylC_{K}-TS and NylC_{A}-TS, respectively (NylC_{K}-TS, Tₘ=87.4° C., NylC_{A}-TS, Tₘ=87.1° C.,, see FIG. 3). As a three-point mutant of NylB′ (NylB homolog from Arthrobacter sp. K172, NylB′-SCY: R187S/F264C/D370Y) was demonstrated to have enhanced PA6 dimer hydrolysis activity, the corresponding mutations were introduced into NylB (from Arthrobacter sp. K172), to generate NylB-SCY, hoping that the mutations would impart additional PA depolymerization activity. The resulting panel of WT and engineered enzymes comprised of 41 candidates. These point mutations are reported in the inventors corresponding journal article (Nature Communications (2024) 15:1217; https://doi.org/10.1038/s41467-024-45523-5), which is, along with its Supplemental Information, Supplementary Data 1, and Supplementary Data 2, incorporated herein by reference in their entireties. Note that the amino acid point mutations identified above and in the published article are off by one location relative what are shown in FIGS. 30-33. This is because the sequences referred to in the journal article each have a start methionine, whereas the sequences illustrated in FIGS. 30-33 do not.

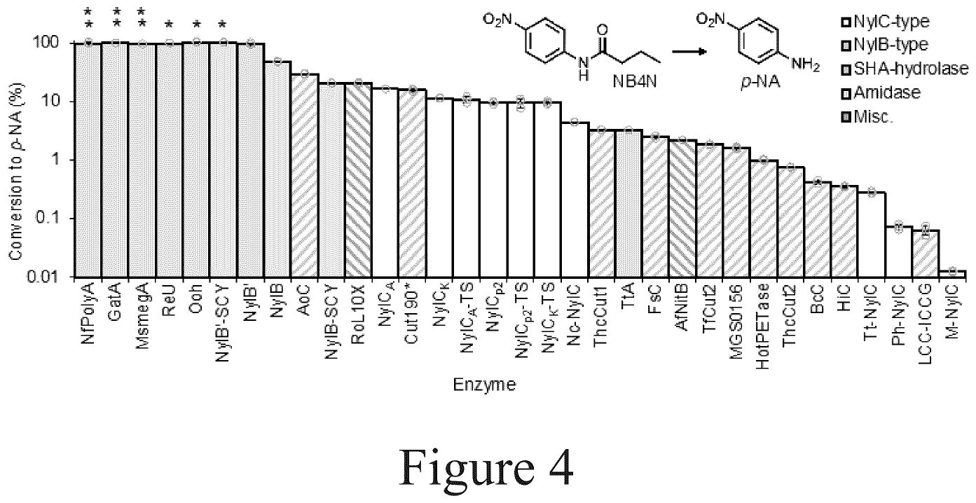

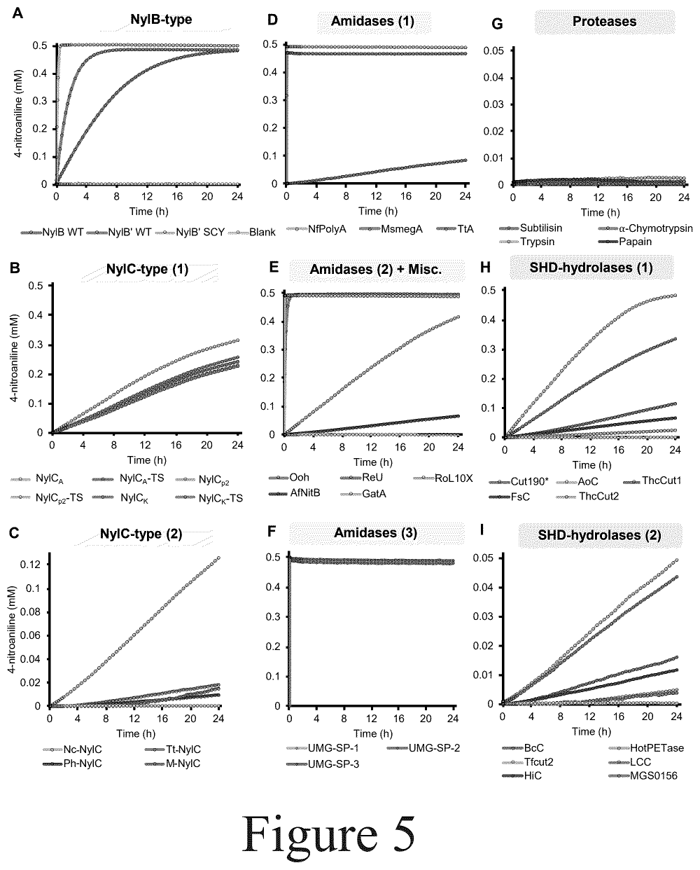

Four enzymes were procured from commercial sources; of the 33 enzymes not available commercially, 32 were successfully expressed and purified from Escherichia coli, to give a 36-member candidate pool. To test the ability of the enzymes for amide bond hydrolysis, each enzyme was incubated with N-butyl-4-nitroaniline (NB4N), a small molecule surrogate that exhibits a butyramide motif akin to the amide bonds in nylon-6 (see FIG. 4 and Panels A-H of FIG. 5). Successful hydrolytic cleavage of the amide bond in NB4N results in the release of p-nitroaniline, which can be monitored spectrophotometrically. Both the amidases (see Panels D and E of FIG. 5) and NylB-type enzymes (sec Panel A of FIG. 5) exhibited the highest levels of amide hydrolysis activity, with NfPolyA (polyamidase from N. farcinica), GatA (unspecified amidase), and MsmegA (metagenomic amidase) completely converting 500 μM NB4N in under five minutes, with ReU (Rhodococcus equi TB-60 urethanase), OoH (ω-octalactam hydrolase) and NylB′-SCY reaching 100% conversion in under 30 minutes. The NylC-type enzymes (see Panels B and C of FIG. 5) and SHA-hydrolases (see Panels G and H of FIG. 5) exhibited a wider range of activities, with the highest performers, NylC_{A} and AoC (Aspergillus oryzae cutinase), displaying 16.2 and 29.3 μM hr⁻¹ p-nitroaniline (p-NA) production, respectively, during the linear phase of the reaction, and the worst performers, M-NylC (Microbacterium sp. NylC) and LCC-ICCG (engineered cutinase from leaf/compost metagenome), only having detectable activity after 8 h and 12 h, respectively. From this small molecule screening, four enzyme candidates (α-chymotrypsin, papain, subtilisin, and trypsin, see Panel F of FIG. 5) were removed from the panel due to no detectable activity under the tested reaction conditions, such that 33 potential nylonases were studied further.

Development of high-throughput solid PA6 depolymerization analysis method by LC-MS/MS: Next the ability of the potential nylonase panel to deconstruct solid PA6 was evaluated. Due to the lack of a strong chromophore in the potential products and the desire for sub part per million (ppm) detection limits, a high-throughput liquid chromatography with tandem mass spectrometry (LC-MS/MS) analysis method was developed to monitor potential major soluble released compounds following PA6 depolymerization reactions. Utilizing tandem mass spectrometry enabled rapid analyses, which reduced the run time. When operating in multiple reaction monitoring (MRM) mode, the instrumentation and detection was optimized for each compound, making the mass transitions highly selective and unique to each analyte, and thus eliminating the need for chromatographic peak separation.

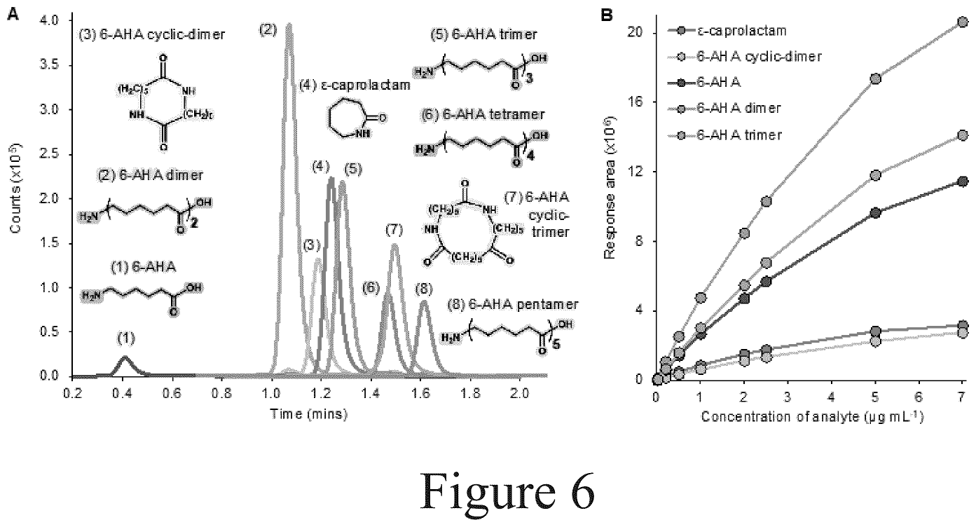

Using this method, linear oligomers of 6-aminohexanoic acid (6-AHA) from monomer to pentamer and cyclic oligomers from caprolactam to the cyclic 6-AHA trimer were all detected in under 3 minutes, representing a significant advancement in the speed and diversity of potential products quantified (see Panel A of FIG. 6). Reaction samples were arrayed across 96-well plates, with a calibration verification standard run every 12 wells to ensure accurate quantification and system stability, demonstrating the suitability of this method for high-throughput analyses. All analytes were detectable from 0.01-7.0 μg mL⁻¹, with samples above the upper quantitation limit further diluted (see Panel B of FIG. 6). The linear 6-AHA from monomer to trimer and cyclic products from caprolactam to 6-AHA cyclic-dimer were quantified using commercial standards. The linear 6-AHA tetramer and pentamer were quantified by applying the calibration curve from 6-AHA linear trimer; the 6-AHA cyclic trimer was quantified utilizing the calibration curve from the 6-AHA cyclic dimer responses.

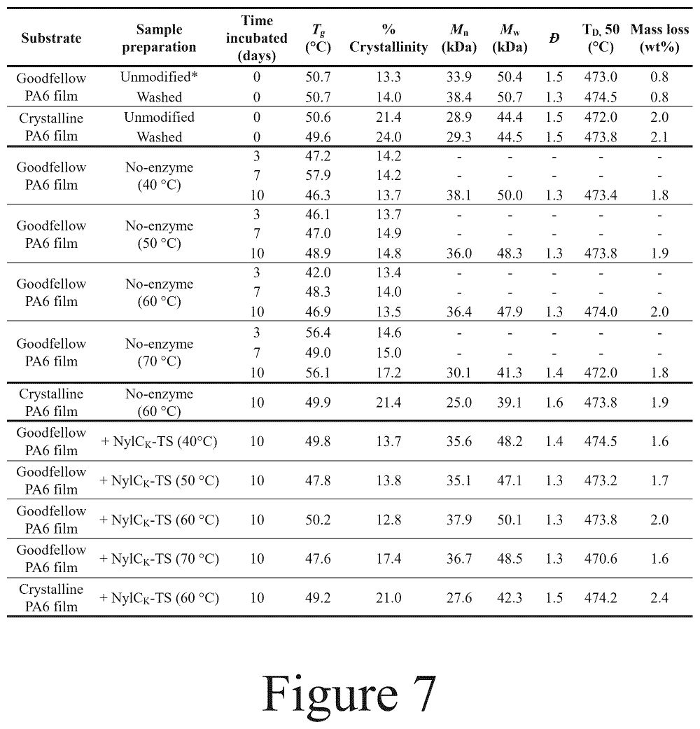

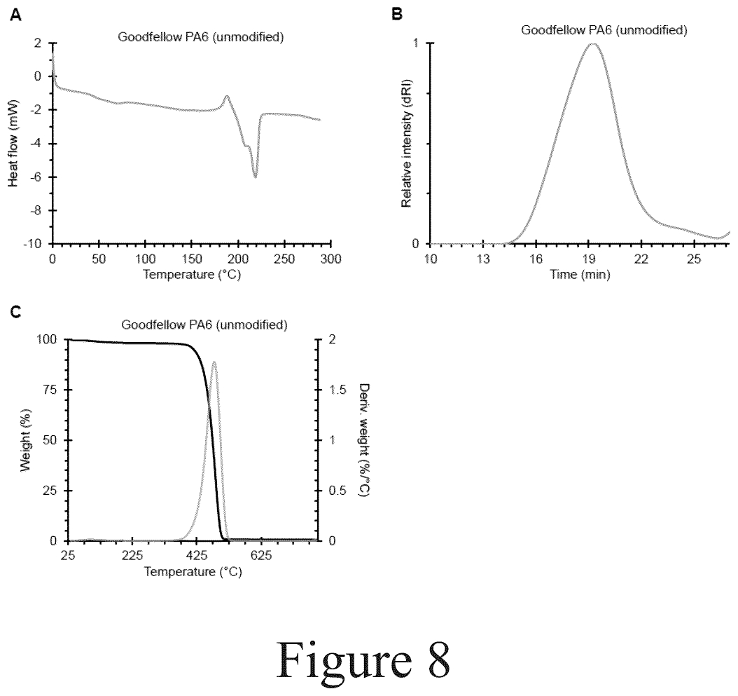

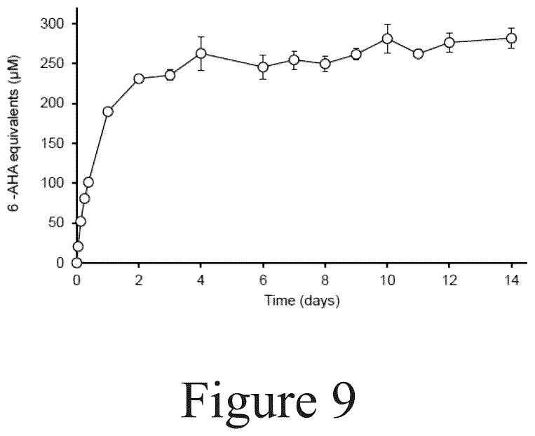

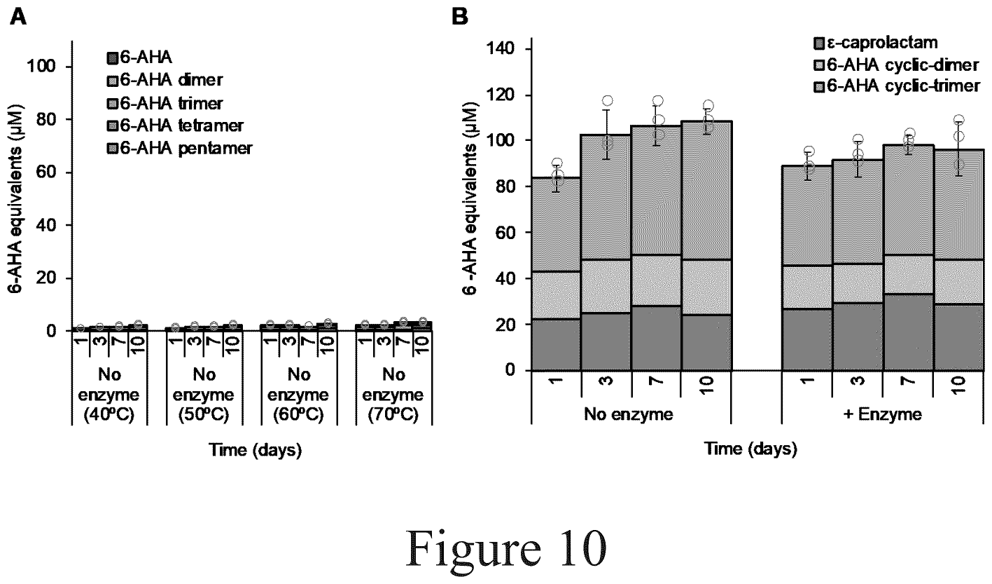

Assessment of potential nylonases on a solid PA6 substrate: With a robust nylon depolymerization analysis method developed, the PA6 deconstruction capabilities of the enzyme panel could be evaluated. For activity screens, commercially available PA6 film from Goodfellow was used as the substrate (13.2% crystallinity, 0.2 mm thickness, full material characterization detailed in FIG. 7, with differential scanning crystallinity (DSC), gel permeation chromatography (GPC) and thermogravimetric analysis (TGA) plots shown in Panels A-C of FIG. 8. PA6 film was washed with DI water prior to use, as detailed in the Methods section, to remove surface-bound cyclic oligomer by-products created during the nylon manufacturing processes. All reactions were conducted for 10 days from 40-70° C., with time points taken at 24, 72, 168, and 240 h (see FIG. 9). Control assays without enzyme led to minimal release of linear 6-AHA oligomers, with the 6-AHA monomer below the limit of detection across all tested temperatures when no enzyme was present (see Panel A of FIG. 10). Enzyme reactions contained 6.5 μM enzyme/mg washed PA6 film and a substrate loading of 0.65 wt % (two squares of 0.5×0.5 cm PA6 film=13 mg) in 100 mM sodium phosphate buffer (NaPi), pH 7.5, 150 mM NaCl. Across all tested enzymes, the extent of ε-caprolactam release was consistent; for most enzymes assayed, concentrations of the 6-AHA cyclic-dimer and cyclic-trimer also remained constant during the reaction (see Panel B of FIG. 10). Hence, for clarity, only the linear products are presented graphically, with cyclic products mentioned only when their levels changed. To aid comparisons of total amount of PA6 depolymerized in each reaction, all products are presented as their 6-AHA equivalents.

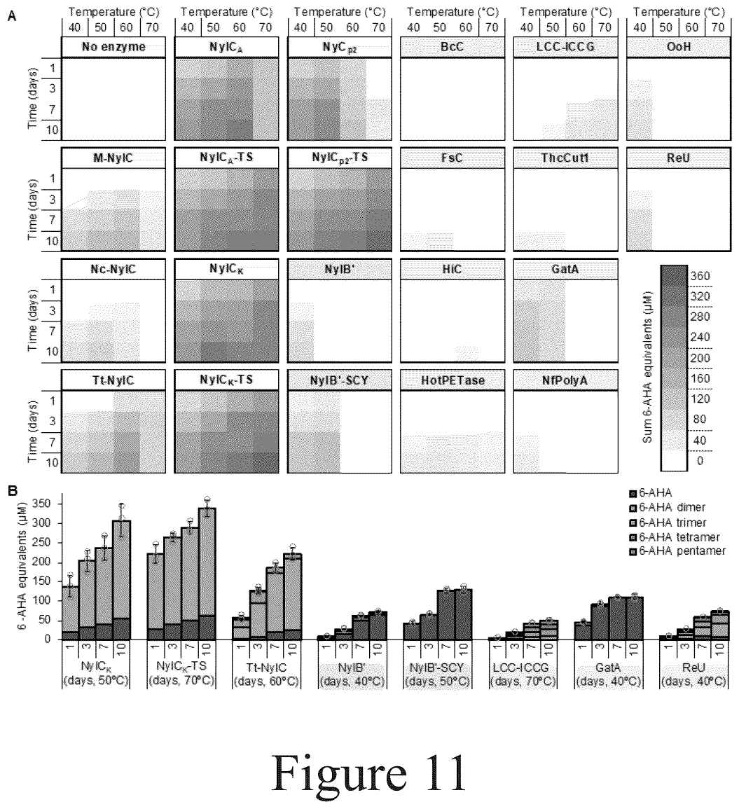

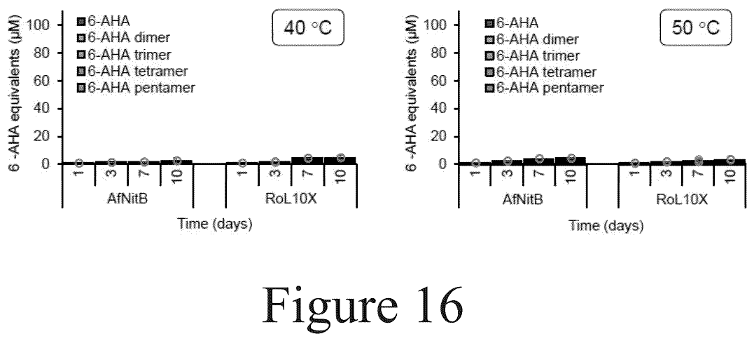

Nylonase activity was observed across a range of enzyme types tested: overall, 26 enzymes demonstrated measurable activity, while five enzymes exhibited no detectable additional soluble product release above background levels (see FIG. 11 and FIGS. 12-16). The temperatures for optimal activity ranged from 40-70° C., with, in general, higher levels of soluble product release seen with increasing temperatures. Interestingly, there appears to be different distributions of products released across the different enzyme groups. Namely, NylB-type enzymes and GatA mainly produce 6-AHA monomer, most amidases and cutinases release a mixture of linear products, with very little 6-AHA pentamer seen with most of the amidases. The most active NylC-type enzyme reactions, however, are dominated by 6-AHA dimer.

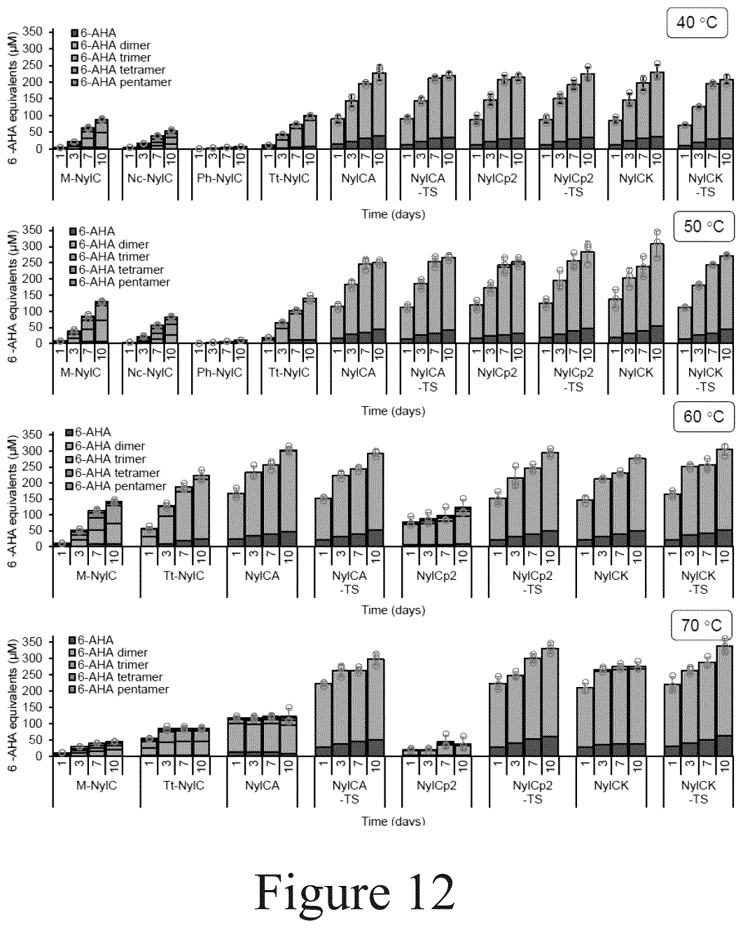

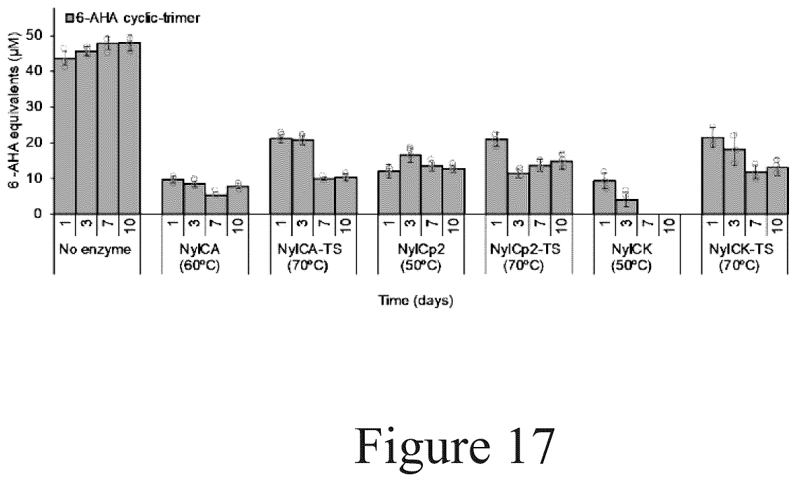

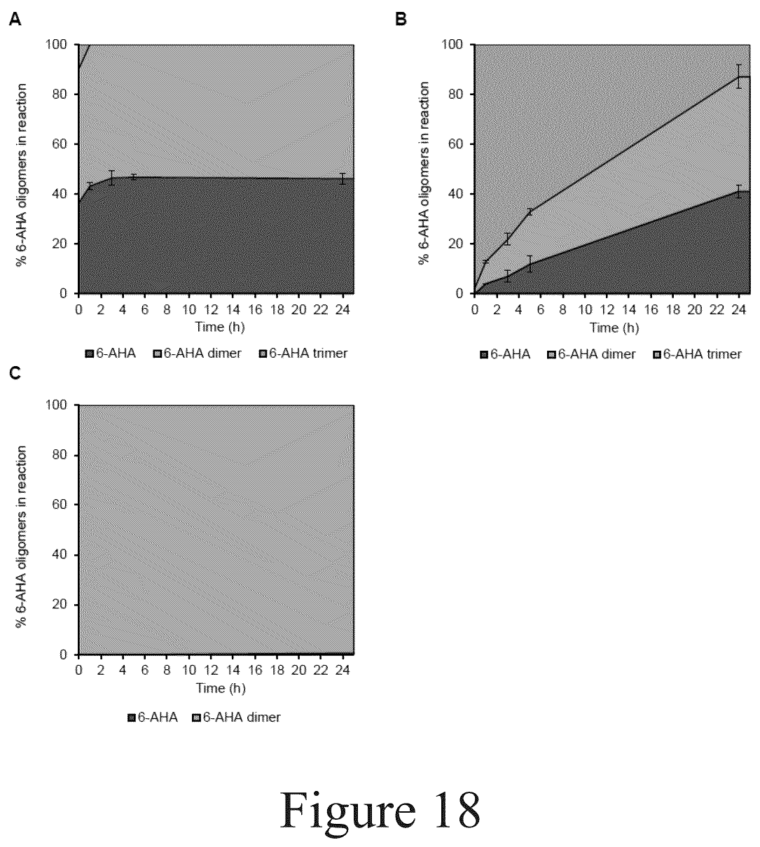

The most active enzymes were from the NylC-type group, with NylCₚ₂, NylC_{A}, NylC_{K}, and their engineered variants identified as the top-performers, being between two and six-fold more active than the best enzymes from other groups. Thermostabilizing mutations appeared beneficial as hypothesized, with all NylC-TS variants showing their optimal activity at 70° C., producing 6-AHA equivalents at a faster rate than their WT equivalents at their optimal temperatures of 50-60° C. (see FIGS. 11 and 12). NylCₚ₂-TS and NylC_{K}-TS additionally showed higher overall product release from 60-70° C. over 10 days compared to the WT enzymes. However, these mutations also appeared to slow the hydrolysis of the 6AHA cyclic-trimer (sec FIG. 17). The significant accumulation of linear trimer in the reactions with the NylC variants found via homology searches is probably a result of their lower propensity for trimer hydrolysis. For instance, NylC_{K}-TS hydrolyzes 100% of 50 μM 6-AHA trimer to dimer and monomer in under 30 minutes at reaction conditions, while trimer hydrolysis is much slower with Tt-NylC (Thermocatellispora tengchongensis NylC), with 81% trimer remaining after five hours (pH 7.5 NaPi buffer, 150 mM NaCl, 2 μM enzyme, 60° C.) (see Panels A and B of FIG. 18). As the 6-AHA dimer remains intact at reaction conditions with NylC_{K}-TS after 24 h (100 μM dimer, pH 7.5 NaPi buffer, 150 mM NaCl, 2 μM NylC_{K}-TS, (see Panel C of FIG. 18), monomer accumulation in NylCₚ₂, NylC_{A}, NylC_{K}, and variant reactions may be due to the fast hydrolysis of released trimers to 6-AHA dimer and monomer. For enzyme NylC_{K}-TS, monomer release at 70° C. after 10 days was about 0.67 wt % depolymerization of the starting mass of PA6.

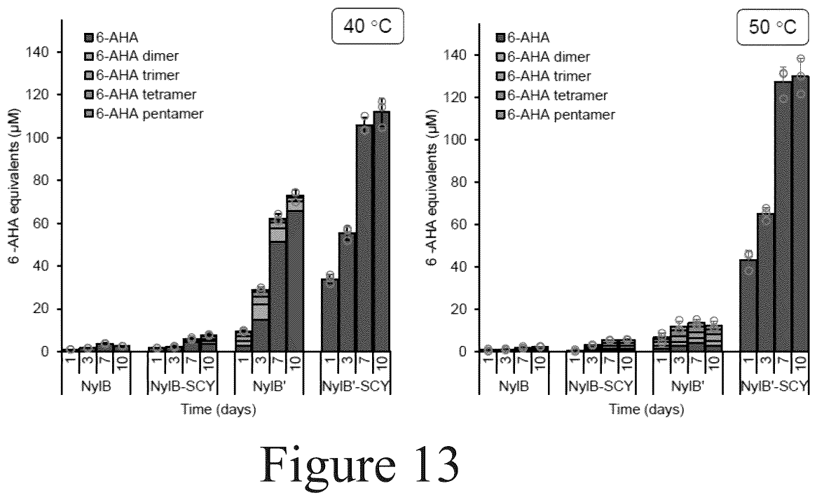

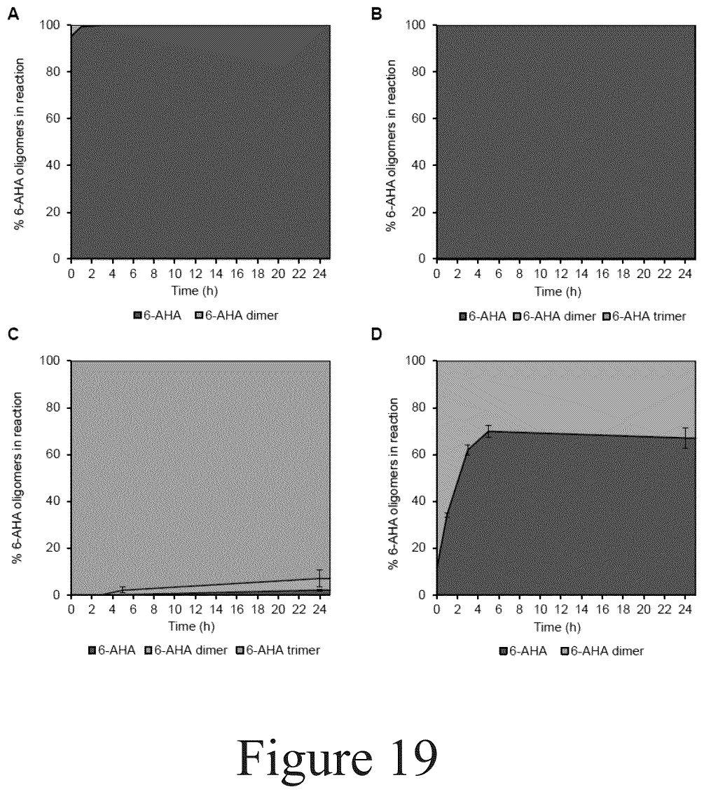

Intriguingly, although NylB appears sparingly active for PA6 film deconstruction, NylB′, a NylB homologue with 88% sequency identity, released 72.9 μM 6-AHA equivalents over 10 days at 40° C. (sec Panel B of FIG. 11 and FIG. 13). The three-point NylB′ variant, NylB′-SCY, is even more active and additionally more thermostable, with optimal 6-AHA release at 50° C., equivalent to 0.26 wt % PA6 deconstruction after 10 days of reaction. Although the NylB′ WT scaffold appears to be the driving force for increased PA6 hydrolysis activity compared to NylB WT, the R187S/F264C/D370Y mutations also appear to have an effect as NylB-SCY is three-fold more active than NylB WT. The total 6-AHA equivalents released over 10 days at 40° C. by NylB-SCY was <8.0 μM. As NylB′-SCY readily hydrolyzes both 6-AHA dimer and trimer at reaction conditions (pH 7.5 NaPi buffer, 150 mM NaCl, 2 μM NylB′-SCY, 50° C.), with 100 μM dimer or 50 μM trimer completely converted to monomer in under 15 minutes (see Panels A and B of FIG. 19), it is difficult from these experiments to identify whether the accumulation of 6-AHA monomer is a product of longer oligomer release followed by fast hydrolysis to monomer, or whether NylB′-SCY exclusively releases 6-AHA from polymer chain ends.

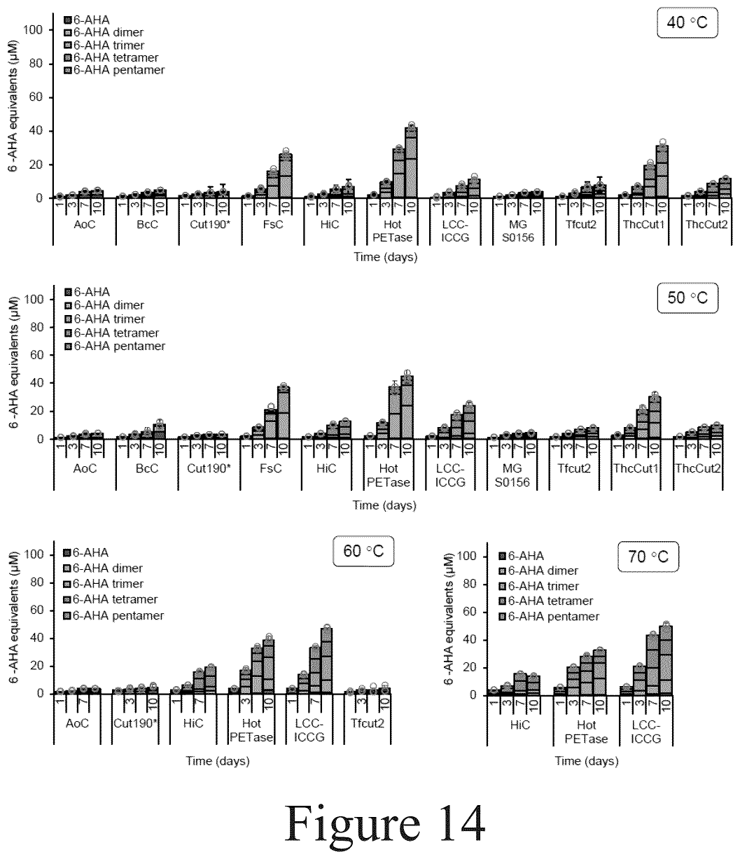

For the nylon-active SHA-hydrolases, Fusarium solani cutinase (FsC) and Thermobifida cellulosilytica cutinase 1 (ThcCut1) have been used previously to modify the surface of PA-6,6 fabric. The reaction profiles and product distributions of all of biocatalysts tested from this group suggest a similar mode of action, with the mixture of soluble oligomers released indicating non-specific cleavage of surface residues, rather than concerted depolymerization activity (see FIG. 14). Support for this mode of action comes from the most active SHA-hydrolase tested, LCC-ICCG (sec Panel B of FIG. 11), which produces only 49.8 μM of 6-AHA over the course of 10 days at 70° C., equating to 0.09% depolymerization of the PA6 film. The observation that mixtures of oligomers are retained over the 10 days of reaction additionally suggests that there is limited further deconstruction of these soluble linear products by SHA-hydrolases. Indeed, incubation of LCC-ICCG with 50 μM 6-AHA trimer for 24 h under reaction conditions (pH 7.5 NaPi buffer, 150 mM NaCl, 2 μM LCC-ICCG, 70° C.), leads to negligible turnover to dimer or monomer (see Panel C of FIG. 19).

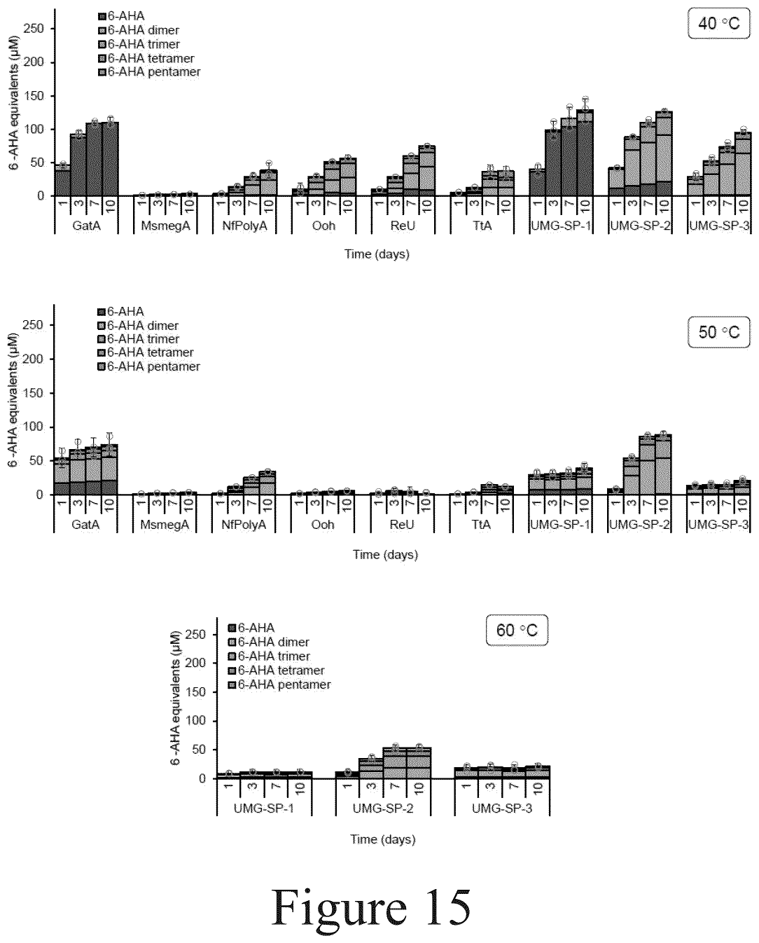

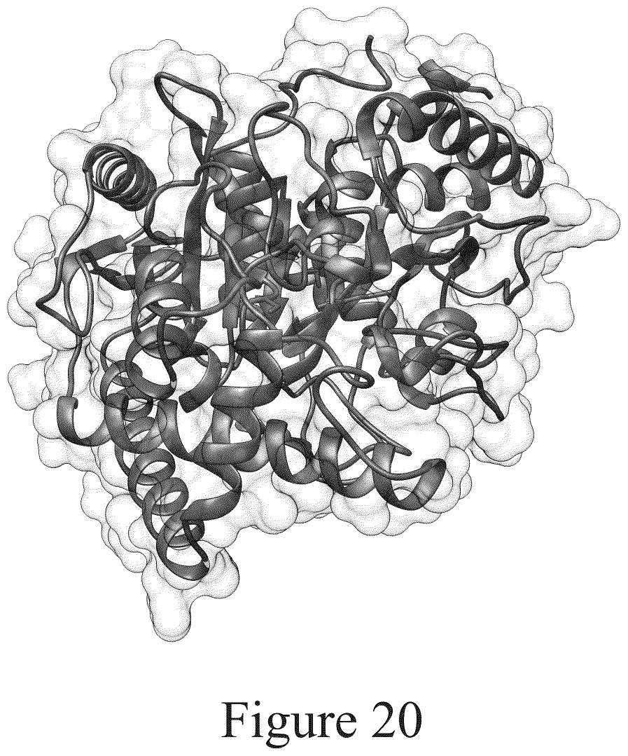

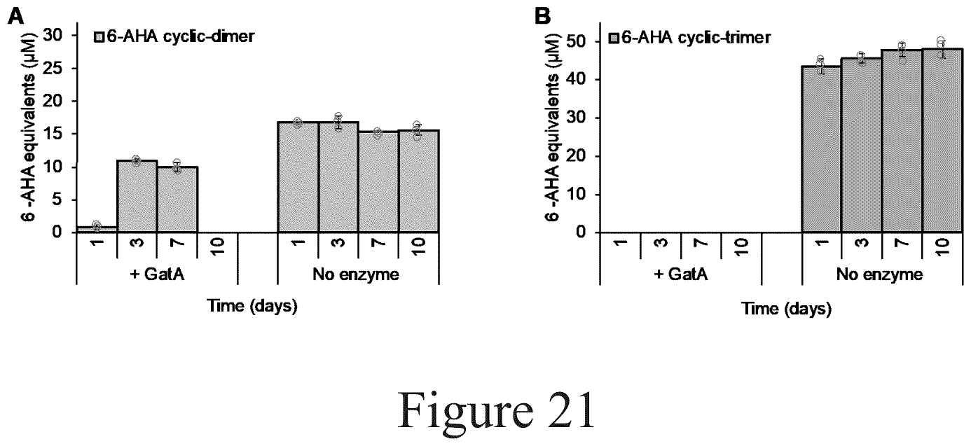

Similarly, low extents of depolymerization are also observed for the amidases, potentially due to their low thermotolerance, with most becoming deactivated above 40° C. (see Panel A of FIG. 11 and FIG. 15). The limited activity of these enzymes was surprising, as from initial screens on a small molecule surrogate substrate, the amidases were among the most active enzymes. However, as the active sites of NfPolyA and its homologues are buried within the enzyme core, their ability to bind and access amide bonds in polymer chains is likely more restricted (see FIG. 20). GatA produced the most 6-AHA equivalents (109 μM of over 10 days at 40° C.) across the amidases (see Panel B of FIG. 11), equivalent to 0.2% PA6 film depolymerization. The interesting preference of GatA for 6-AHA monomer release is in part due to its nylon oligomer hydrolysis ability; namely, GatA can hydrolyze linear products, with 67% of 100 μM 6-AHA dimer turned over to monomer under reaction conditions (pH 7.5 NaPi buffer, 150 mM NaCl, 2 μM GatA, 40° C.) within 24 h (see Panel D of FIG. 19), but it also hydrolyzes cyclic oligomers. Unlike any other enzyme tested here, GatA completely depletes both 6-AHA cyclic-dimer and trimer during PA6 film reactions, indicating that a proportion of the monomer release witnessed is the product of hydrolysis of these surface contaminants (see FIG. 21). As with the SHA-hydrolases, the low rates of depolymerization for all the amidases again suggests a surface modification process rather than significant bulk PA6 depolymerization.

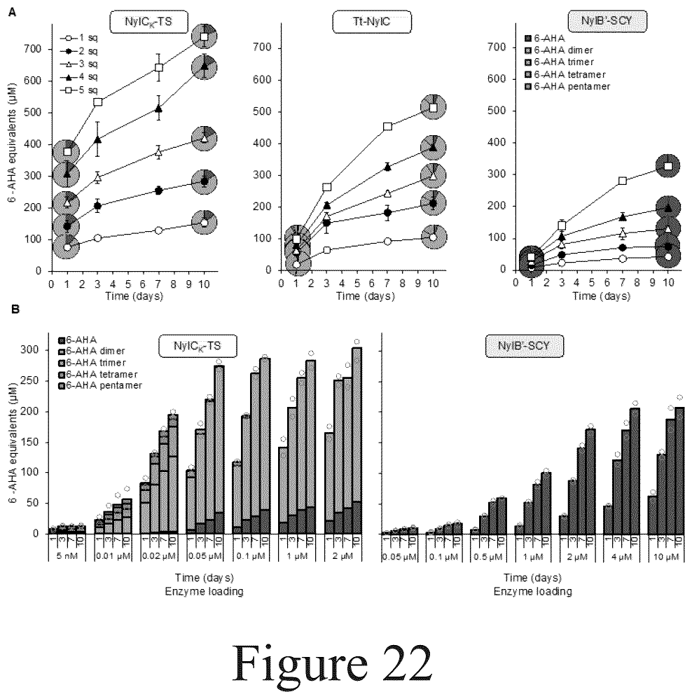

In-depth characterization of the best performing enzymes: A subset of the best performing enzymes were examined, including NylC_{K}-TS, Tt-NylC, and NylB′-SCY, to test if the rate and extent of PA6 depolymerization could be improved and the mode of action understood. NylB′-SCY reactions were carried out at 50° C., its highest operating temperature, while NylC-type enzyme reactions were conducted at 60° C. to allow fair comparison between homologues. For all three enzymes, increasing the substrate loading from 0.32-1.6 wt % (one to five squares PA6 film, 1 μM enzyme, 100 mM sodium phosphate buffer (NaPi), pH 7.5, 150 mM NaCl) lead to greater product release, most likely due to an increase in available reactive surface area (see Panel A of FIG. 22). Product distributions remained constant for both NylC_{K}-TS and NylB′-SCY at all substrate loadings, while the 6-AHA trimer accumulates in Tt-NylC depolymerizations.

Increasing enzyme loading (13 mg PA6 film (0.65 wt % substrate loading), 100 mM sodium phosphate buffer (NaPi), pH 7.5, 150 mM NaCl), surprisingly, had little effect on total PA6 film depolymerization (see Panel B of FIG. 22). 6-AHA equivalent release never surpassed 300 μM under any condition, with improvements in total NylC_{K}-TS depolymerization stalling above 0.325 μM enzyme/mg PA6 (0.18 mg enzyme/g PA6). However, at low NylC_{K}-TS enzyme loadings, where the reaction rate will be slowed, a higher proportion of 6-AHA trimer to pentamer products was observed. A proportion of NylC_{K}-TS PA6 hydrolysis events may therefore be random, producing mixed length oligomers that are subsequently hydrolyzed to dimer and monomer. Slowed oligomer hydrolysis in low enzyme loading reactions additionally suggests a preference of NylC_{K}-TS for release of product from the polymer surface over hydrolysis of oligomers in solution. Reactions with Tt-NylC support this hypothesis as trimer levels were only reduced at very high enzyme loadings. These trends, however, are not observed for NylB′-SCY: even at the lowest enzyme loading, only 6-AHA is produced. As an important consideration for further application, significantly less NylC_{K}-TS was required to achieve the highest total depolymerization (0.05 μM NylC_{K}-TS, 0.18 mg enzyme/g PA6), compared to both Tt-NylC or NylB′-SCY (10 μM enzyme, 53.8 mg enzyme/g PA6 and 125 mg enzyme g/PA6, for Tt-NylC and NylB′-SCY, respectively).

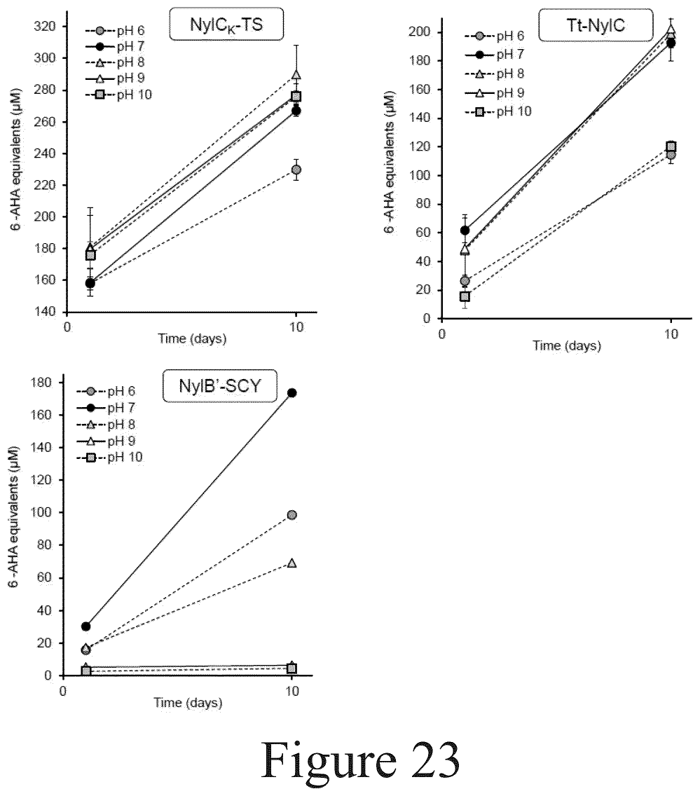

Variation of reaction pH from pH 6-10 (3.25 μM enzyme/mg PA6, substrate loading 0.65 wt %, 100 mM buffer, 150 mM NaCl) also did not elicit higher levels of PA depolymerization, however it did reveal differences in pH tolerance amongst the three enzymes (sec FIG. 23). NylC_{K}-TS was mostly unaffected by pH changes, with Tt-NylC being similarly pH-robust with drops in activity only seen at the extremities of the pH range tested. Conversely, NylB′-SCY is particularly sensitive to pH changes: pH 7 is optimal with significant decreases in activity either side of this and complete deactivation from pH 9-10.

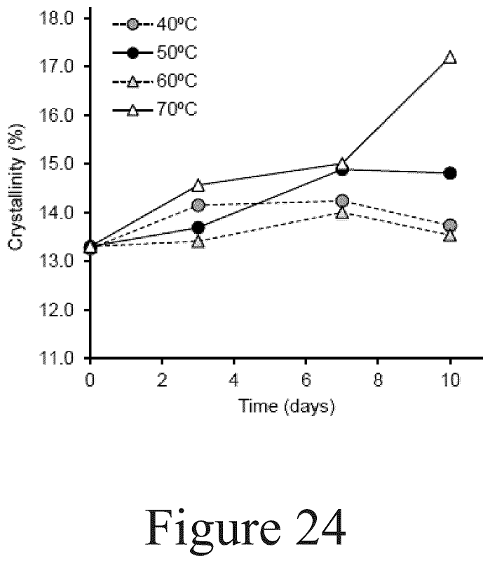

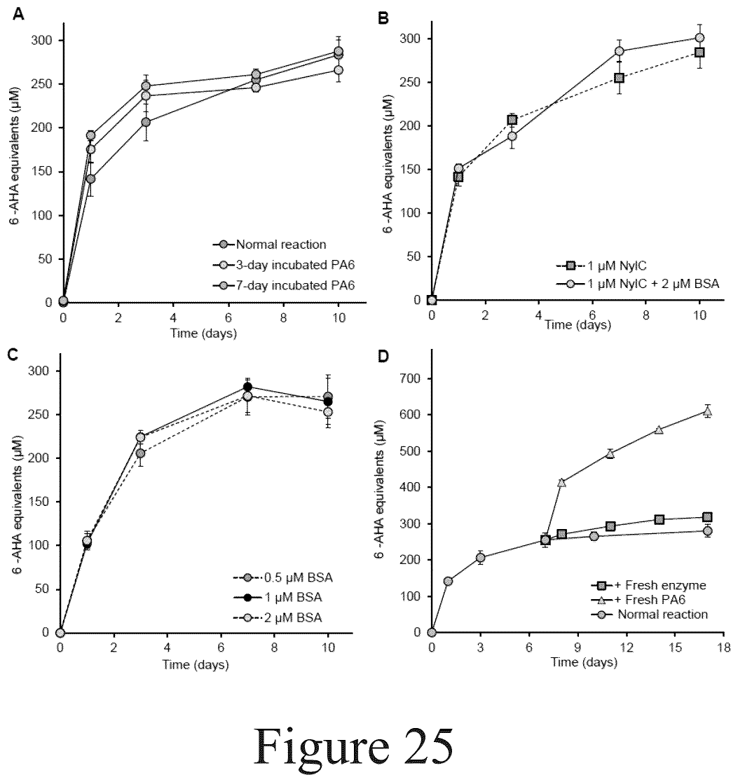

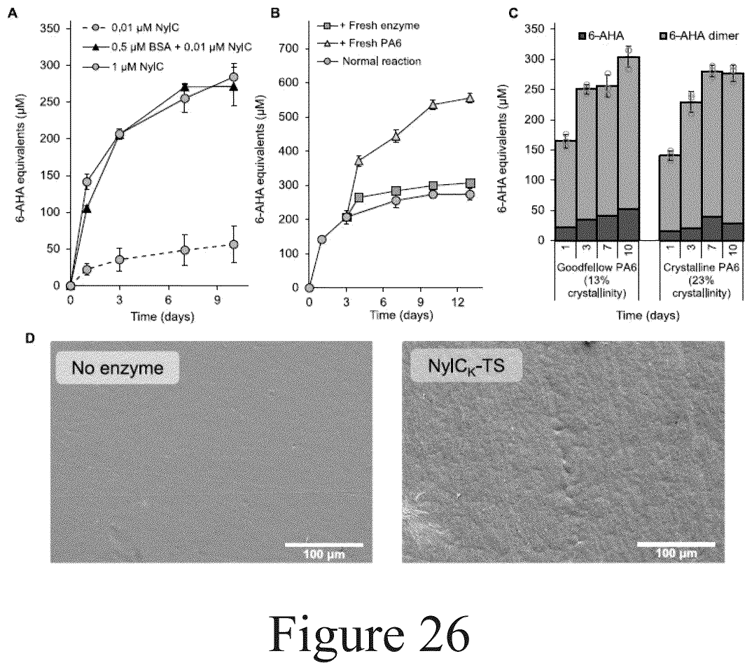

Examination of the reaction profile of NylC_{K}-TS: Next, identification of what was limiting enzymatic PA6 depolymerizations and causing the observed asymptotic reaction profiles was evaluated, using the most active enzyme, NylC_{K}-TS, as the test case. Slight crystallinity increases in the PA6 substrate were identified in no-enzyme control reactions in reaction buffer (100 mM sodium phosphate buffer (NaPi), pH 7.5, 150 mM NaCl) at 70° C. (maximum 3.2% increase in crystallinity over 10 days), hence, reactions were carried out at 60° C. where this effect was less noticeable (maximum 0.2% increase in crystallinity over 10 days) (see FIGS. 7 and 24). Reactions contained 3.25 μM enzyme/mg PA6 and 13 mg PA6 (substrate loading of 0.65 wt %), in 100 mM pH 7.5 NaPi buffer, 150 mM NaCl. To rule out effects of incubation-induced PA6 changes during the reaction, we conducted NylC_{K}-TS depolymerizations using PA6 film incubated in reaction buffer at 60° C. for either three or seven days prior to enzyme addition. As there was no significant difference between the product release of pre-incubated PA6 film versus non-incubated PA6, this could be discounted (see Panel A of FIG. 25). Interestingly, while not promoting additional depolymerization, including the non-specific binding inhibitor, bovine serum albumin (BSA) in reactions allows for the same level of PA6 depolymerization with 100-fold less enzyme (0.0325 μM NylC_{K}-TS/mg PA6, substrate loading of 0.65 wt %, 0.5 μM BSA, 100 mM NaPi buffer, 150 mM NaCl, pH 7.5, 60° C. (see Panel A of FIG. 26 and Panel B of FIG. 25), suggesting that while NylC_{K}-TS may be affected by non-specific binding events, this is not a main factor limiting depolymerization extent. Increasing concentrations of BSA further did not enhance PA6 deconstruction levels (see Panel C of FIG. 25).

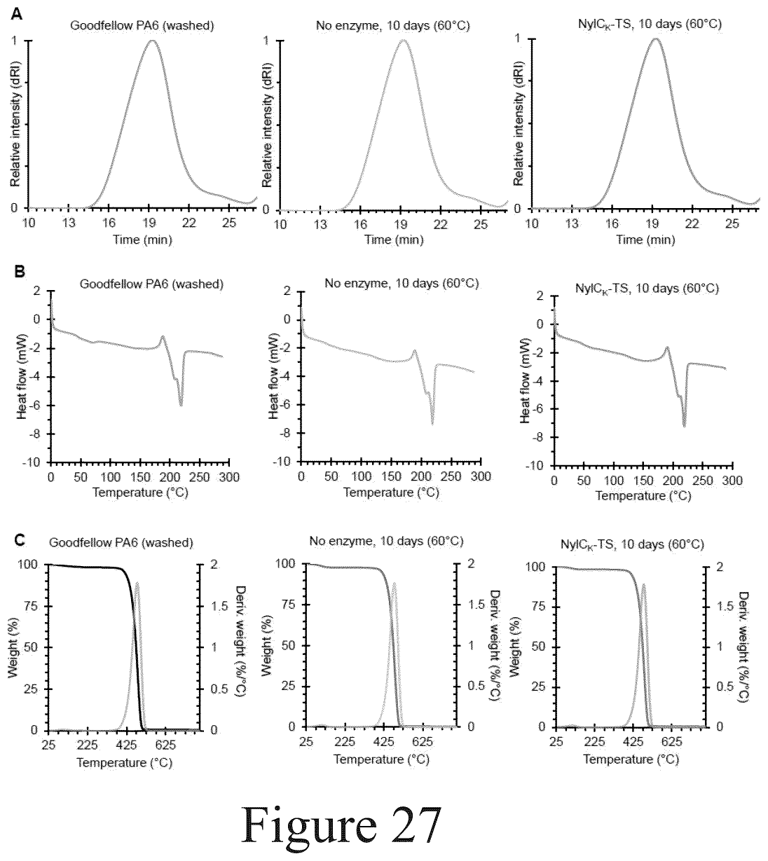

Reaction progress was not recovered by supplementation with fresh NylC_{K}-TS after either three or seven days of enzyme reaction (see Panel B of FIG. 26 and Panel D of FIG. 25). However, addition of new PA6 substrate (two squares) to a NylC_{K}-TS depolymerization reaction that had been running for three days (the start of reaction plateau), led to additional product release of a similar magnitude from the first substrate, with the same relationship seen after substrate addition to a seven-day reaction (see Panel B of FIG. 26 and Panel D of FIG. 25). Reaction profiles following new substrate addition match 6-AHA equivalent release seen in standard reactions like those shown in FIG. 22, indicating that NylC_{K}-TS retains almost full activity after both three and seven days of reaction at 60° C., and there are no inhibitory compounds present that could explain reaction stalling. Materials characterization analysis suggests there was no extensive change in the average molecular weight (Mₙ) or polydispersity (PDI) of the PA6 polymer chains following 10 days of enzyme incubation with NylC_{K}-TS at any temperature (see FIG. 7 and Panel A of FIG. 27), nor were there any significant differences in the percentage crystallinity of the PA6 substrate incubated with or without enzyme over the course of 10 days (see Panel B of FIG. 27). As a note, TGA analysis revealed that the PA6 film absorbed an average of 1.9 wt % water following 10 days incubation in reaction buffer (no enzyme control), and there was no noticeable difference in substrate water absorption in reactions with NylC_{K}-TS (average of 1.7 wt %) sec FIG. 7 and Panel C of FIG. 27).

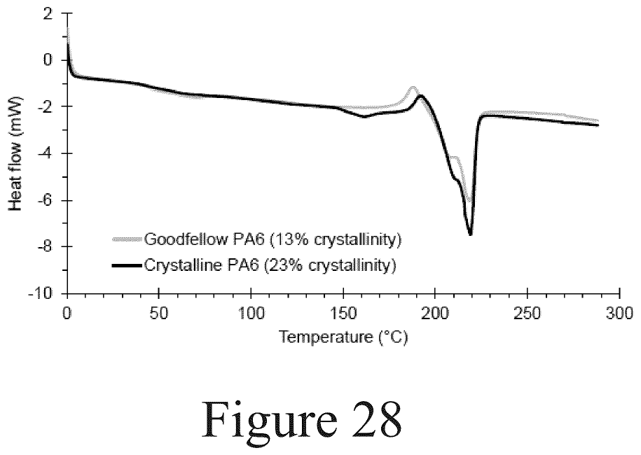

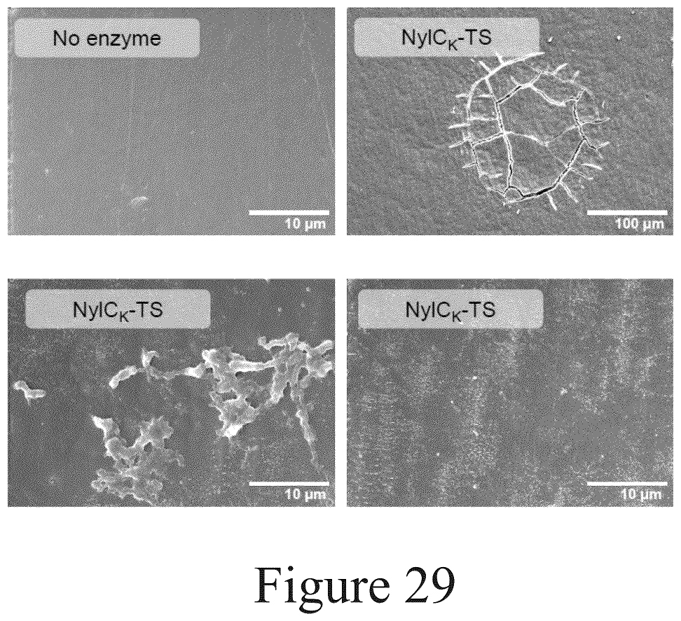

Taken together, these results suggest that the reaction plateau for NylC_{K}-TS is a consequence of lack of remaining hydrolysable substrate for this enzyme following 10 days of reaction. Furthermore, usually for more extensive enzymatic plastic depolymerizations, both rate and extent of deconstruction are highly sensitive to substrate crystallinity. However, for PA6 depolymerization with NylC_{K}-TS, using a more crystalline PA6 film substrate (23% crystallinity, synthesized in house, full material characterization in FIG. 7 and FIG. 28), leads to an equivalent amount of 6-AHA equivalent release compared to reactions with the Goodfellow film that is more amorphous (13.2% crystallinity, see Panel C of FIG. 26. Hence, one can conclude that NylC_{K}-TS is likely only working on a small amount of accessible nylon polymer on the film surface, and so does not reach the depolymerization extents at which substrate crystallinity would play a role in reaction progression. Indeed, SEM images of PA6 incubated with NylC_{K}-TS (3.25 μM enzyme/mg PA6, 0.65 wt % substrate loading, 100 mM pH 7.5 NaPi buffer, 150 mM NaCl, 60° C., 10 days), reveal a slight surface roughening, but no significant pits or features that are commonly associated with more extensive biocatalytic polymer deconstruction (see Panel D of FIG. 26 and FIG. 28).

In summary, the PA6 deconstruction capacity of a diverse panel of potential nylonases was studied using a high-throughput LC-MS/MS-based analysis strategy. This work demonstrates that enzymes from a wide variety of sources exhibit sparing capacity to release soluble oligomeric products from a PA6 film. Enzymes with the canonical Ser-His-Asp catalytic triad and the amidases were shown to exhibit low propensities for PA6 depolymerization. There was little correlation between the capacity for an enzyme to hydrolyze the soluble NB4N substrate relative to solid PA6 deconstruction.

NylB-type enzymes demonstrate that they can act on solid PA6, with NylB′-SCY being the most active (0.26% PA6 film depolymerized, 125 mg enzyme/g PA6). The wide difference in activity between NylB and NylB′ for PA6 film hydrolysis was surprising, as NylB is 200 times more active for 6-AHA dimer hydrolysis than NylB′. With 6-AHA monomer being the sole product under all conditions, it may be that NylB′-SCY is acting in exo-type manner, cleaving terminal monomer moieties from the PA6 film surface, or that the hydrolysis of released longer oligomers is too fast to observe. In either case, the release of a homogenous reaction product may be desirable for downstream applications, such as recircularization of 6-AHA to form caprolactam for new nylon production.

The rationally mutated NylC_{K}-TS variant exhibited the highest extents of depolymerization, indicating that thermostability is indeed a useful feature. Introduction of just two mutations led to an increase in Tₘ of 27.1° C. (NylC_{K}-TS Tₘ=87.1° C.), indicating that the enzyme is highly engineerable. NylC_{K}-TS proved very stable, retaining activity after even seven days of reaction at 60° C. and required the lowest enzyme loadings to achieve the highest levels of PA6 depolymerization (0.18 mg enzyme/g PA6), both desirable characteristics of an industrial biocatalyst. NylC_{K}-TS appears to cleave the polymer in a non-specific manner, with a mixture of soluble oligomers initially released before further hydrolysis, likely through a non-processive mechanism of action. Drawing parallels with PET hydrolysis, it may be that enzymatic PA6 deconstruction proceeds via a combination of endo- and exo-lytic bond cleavages. As NylC-type enzymes all produced 6-AHA dimer as the major product, if these biocatalysts are selected for further application, it may be that a two-enzyme system is needed to further hydrolyze the dimer to create a single 6-AHA product. It is most likely that NylC_{K}-TS, along with the other enzymes studied, currently deconstructs only on a small amount of easily accessible polymer, being unable to progress further once this has been removed, suggesting that a polymer pretreatment step to encourage further depolymerization may be necessary.

### Methods

Materials: PA6 film (0.2 mm thick, 13.2% crystallinity, full material characterization available in FIG. 7 and FIG. 8 was sourced from Goodfellow (product ID: AM30-FM-000200).

Crystalline PA6 film (0.2 mm thick, 23% crystallinity, full material characterization available in FIG. 7 and FIG. 28 was prepared by cutting up Goodfellow PA6 film into smaller pieces and drying at 150° C. in a vacuum oven overnight. The pieces were hot pressed between Teflon sheets at 250° C. for 1 min at 1,000 psia. The Teflon/nylon film assembly was then placed on a pre-heated hot plate at 200° C. under a pre-heated steel block for 10 min. Finally, the film was dried in a vacuum oven at 200° C. for 2 h.

Prior to use, all PA6 films were cut into 0.5 cm×0.5 cm squares and washed in DI water for 3 h at 37° C., rinsed in DI water once and then dried.

All reagents, chemicals and buffer components were sourced from Sigma-Aldrich, unless specified otherwise. HiC was purchased from Novozymes (Novozym® 51032), subtilisin A, trypsin, and papain from Sigma-Aldrich, and α-chymotrypsin from Worthington Biochemical, USA. Butyric anhydride was sourced from TCI chemicals and 1,1,1,3,3,3-hexafluoroisopropanol from Chem IMPEX.

Enzyme identification and homology searches: Literature searches were conducted to find enzymes with observed activity on solid PA6, PA6,6, PA6 oligomer, or polyurethane (PUR), considering both scientific reports and patents. As ROL-WT could not be expressed in E. coli, a previously described mutant which had previous successful E. coli expression (RoL10X) was used. The Ser-His-Asp hydrolase group of enzymes was selected from an in-house PET hydrolase library, including two previously reported thermostabilized variants, LCC-ICCG and HotPETase. Homology searches for additional NylC-type enzymes and amidases were carried out using UniProt BLAST, with NylCₚ₂ and NfPolyA as the input protein sequences, using the suggested preset paramaters. From the output sequence homology lists, several candidates were selected that came from potentially thermophilic microorganisms. The phylogenetic tree was created using Maximum Likelihood analysis, with 1,000 bootstraps; the data were visualized as a phylogenetic tree using the Interactive Tree of Life (iTOL) online tool.

Rational mutagenesis of NylCs and NylB: Rational mutations for increasing the thermostability of NylC_{A} and NylC_{K} were selected based on positions identified by rational mutagenesis of NylCₚ₂ for thermostability by Negoro et al. The Negoro 4-point mutant, NylCₚ₂-TS, (NylCₚ₂-D36A/D122G/H130Y,/E263Q) has an increased Tₘ of 88° C. (36° C. increase over the WT). As Negoro et al. found that mutating G111 and L137 of NylCₚ₂ to the analogous residues in NylC-_{K} and NylC_{A} (S111 and A137) significantly decreased thermostability, we hypothesized that the G111 and L137 from NylCₚ₂ may be a more stable configuration of these interacting residues, and hence we introduced them into the scaffolds of NylC_{K} and NylC_{A}. In addition, the E263Q mutation was included in NylC_{A} as this change additionally increased the thermostability of NylCₚ₂ in the context of D122 and H130, which NylC_{A} already possesses. This gave the thermostabilized variants NylC_{K}-TS (NylC_{K}-S111G/A136L) and NylC_{A}-TS (NylC_{K}-S111G/A136L/E263Q). The NylB mutation was informed by previous work on the NylB homologue, NylB′. A three point mutant, NylB′-SCY (NylB′ R187S/F264C/D370Y), had enhanced 6-AHA dimer hydrolysis ability as compared to the wildtype NylB′ enzyme, hence, S187/C264/Y370 were introduced at the analogous positions in NylB to give NylB-SCY.

Plasmid construction: All protein-encoding genes were codon optimized for expression in E. coli, and inserted into either pET-21b, pET-28a or pET-29b expression vectors between NdeI (5′ end) and XhoI (3′ end) restriction sites, dependent on the enzyme. All vectors contain a hexa-histidine tag coding sequence for downstream protein purification. Plasmids were synthesized by Twist Biosciences.

Enzyme expression: All enzymes were expressed in chemically competent BL21 E. coli cells. Single colonies of freshly transformed cells were grown at 37° C. overnight in 5 mL of LB medium supplemented with the appropriate antibiotic for the expression vector (100 μg mL⁻¹ ampicillin or 25 μg mL⁻¹ kanamycin). 2 mL of the resulting culture was used to inoculate 50-100 mL of 2YT medium containing either 100 μg mL⁻¹ ampicillin or 25 μg mL⁻¹ kanamycin, as appropriate. Cultures were grown at 37° C., 190 rpm, to an OD₆₀₀ of 1. Protein production was initiated by the addition of IPTG (final concentration of 0.1 mM), and cultures grown for a further 20 h at 20° C. Cells were collected by centrifugation at 3,220 g for 10 mins and stored at −20° C. until purification.

Enzyme purification: Cell pellets were resuspended in lysis buffer (pH 7.5, 50 mM Tris-HCl, 10 mM imidazole, 300 mM NaCl, 1.0 mg mL⁻¹ lysozyme, 10 μg mL⁻¹ DNasc) and subjected to sonication. The resulting lysate was clarified by centrifugation (13,500 g for 15 mins) and the soluble fraction applied to Ni-Nta agarose (Anatrace). Unbound proteins were removed with lysis buffer, followed by elution of bound proteins with elution buffer (pH 7.5, 50 mM Tris-HCl, 300 mM imidazole, 300 mM NaCl). Proteins were the applied to 10DG desalting columns (Bio-Rad) and eluted in reaction buffer (100 mM sodium phosphate buffer (NaPi), pH 7.5, 150 mM NaCl). Protein purity was confirmed by SDS-PAGE, with concentrations determined by 280 nm absorbance readings.

Enzyme thermostability analysis: The thermostabilities of the NylC-TS variants were determined using differential scanning fluorimetry (DSF). For each protein, a 5 μM sample of protein was prepared in reaction buffer and SYPRO Orange dye added to a final concentration of 10×. DSF was carried out using a Bio-Rad CFX Connect 96-Real Time PCR system, using the FRET channel for excitation and emission settings. The temperature was increased with an increment of 0.3° C. s⁻¹ from 25° C. to 95° C. The Tₘ was then determined from the peak of the first derivative of the melt curve from three replicate measurements.

Screening for activity on NB4N: Activity screens on N-butyl-4-nitroaniline (NB4N) were conducted as follows: a reaction mixture of 500 μM NB4N in reaction buffer was arrayed in a 96-well plate. To initiate the reaction, 2 μM of each enzyme was added, the plate sealed with optically clear film, and the release of p-nitroaniline (pNA) followed by change in absorbance at 410 nm monitored using a Biotek Synergy HT plate reader over the course of 24 h at 30° C. The concentration of pNA was determined by constructing a calibration curve. Enzyme reactions were carried out in triplicate.

Screening for enzyme activity on PA6 film: The initial activity screens on PA6 films for all enzymes were carried out as followed: reactions contained two squares of PA6 film (13.19 mg±0.13 mg PA6) in reaction buffer (100 mM sodium phosphate buffer (NaPi), pH 7.5, 150 mM NaCl). 2 μM of enzyme was added to initiate the reaction, giving a final reaction composition of 6.5 μM enzyme/mg PA6 film and a substrate loading of 0.65 wt %. Reactions were carried out in triplicate at 40, 50, 60, and 70° C. over the course of 10 days at 180 rpm, with samples taken at 24, 72, 168, and 240 hs. For the characterization of the best performing enzymes, reactions were conducted at 50° C. for NylB′-SCY and 60° C. for Tt-NylC and NylC_{K}-TS to maximize reaction yield while avoiding potentially confounding effects of thermally induced PA6 crystallization observed at higher temperatures. Reactions with variable enzyme or substrate loadings were also carried out in an analogous manner to the initial activity screens with the following changes: for variable enzyme loadings, enzyme concentration was varied from 0.005 μM up to 10 μM per reaction using two squares of PA6 film as the substrate, while for changing substrate loadings, 1 μM of enzyme was applied to one to five squares of PA6 film per reaction. For reactions with changing pHs, all buffers were made to 100 mM and supplemented with 150 mM NaCl, using citrate buffer at pH6, NaPi buffer at pH 7-8 and glycine-OH buffer from pH 9-10, and each reaction contained 1 μM of enzyme and 2 squares of PA6, equating to a final reaction composition of 3.25 μM enzyme/mg PA6 film and a substrate loading of 0.65 wt %. All time point samples were quenched in 5× MeOH and stored at 4° C. prior to analysis. The extent of PA6 film depolymerization by mass was calculated from the release of soluble PA6 depolymerization products as determined by LC-MS/MS.

Activity assays with nylon oligomers: Activity assays with 6-AHA dimer and 6-AHA trimer were carried out as follows: 50 μM of 6-AHA trimer or 100 μM 6-AHA dimer were placed in reaction buffer, pH 7.5 NaPi, 150 mM NaCl. Reactions were initiated by addition of 2 μM enzyme and incubated at the optimal reaction temperature of the described enzyme. Reactions were monitored over the course of 24 h. No-enzyme controls showed no detectable hydrolysis of either 6-AHA trimer or 6-AHA dimer over 24 h at reaction conditions.

Characterization of NylC_{K}-TS activity: For additional characterization of NylC_{K}-TS activity, reactions contained 1 μM enzyme and two squares of PA6 film in reaction buffer and were incubated at 60° C. unless otherwise stated, giving a final reaction composition of 3.25 μM enzyme/mg PA6 film and a substrate loading of 0.65%. Crystalline PA6 reactions were carried out in the same manner using two squares of PA6 crystalline film as the reaction substrate. For reactions with pre-incubated substrate, two PA6 film squares were incubated in reaction buffer for either three or seven days, prior to addition of 1 μM enzyme. For reactions with fresh enzyme added, a NylC_{K}-TS activity assay was carried out for either three or seven days with a time-point sample taken for analysis at the end of reaction, the PA6 squares were removed, sonicated in DI water, rinsed and placed in fresh reaction buffer, 1 μM of fresh enzyme was then added and the reaction allowed to progress with additional product formed added to the value of the time-point sample taken at the end of the first reaction. For reactions with fresh PA6 substrate added, a NylC_{K}-TS activity assay was carried out for either three or seven days with a sample taken for analysis of T₀, two new squares of PA6 were then added to the same reaction and reaction progress monitored further. For reactions with bovine serum albumin (BSA), reactions contained 0.01 μM NylC_{K}-TS, tow squares of PA6 film (0.33 μM enzyme/mg PA6 film and a substrate loading of 0.65 wt %) and 0.5 to 2 mM BSA.

PA6 film characterization: Films were prepared in their “raw” state, as obtained from the manufacturer, “washed” state, following washing in DI water prior to reactions, or their “incubated” state where films were incubated in reaction buffer under the same conditions as an enzymatic assay from 40-70° C. Films were also assessed after enzyme reactions, following incubation with 1 μM NylC_{K}-TS at 60° C. for 10 days. Prior to analysis, other than the unmodified “raw” films, all samples were rinsed in DI water to remove salts and enzymes. The thermal stability of nylon samples was assessed by thermogravimetric analysis (TGA) using a Discovery TGA 5500 (TA Instruments). For each run, 6-10 mg of polymer sample was placed in a platinum TGA pan. Samples were heated under nitrogen from ambient temperature to 200° C. at a rate of 4° C. min⁻¹, then from 200° C. to 800° C. at a rate of 20° C. min⁻¹. TRIOS software (TA Instruments) was used to characterize the onset temperature of polymer degradation (T_{D,5}) and the weight percent of residual char at 800° C. The mass percent loss and temperature of the derivative maximum were determined for each mass loss event.

Differential scanning calorimetry (DSC) was used to measure the thermal properties and crystallinity of raw and treated nylons. Samples were dried at 40° C. for 24 h in a vacuum oven to remove absorbed water immediately prior to analysis. DSC measurements were performed on a Discovery X3 Differential Scanning calorimeter (TA Instruments) using 5-8 mg of sample in hermetically sealed aluminum pans (DSC Consumables). Each DSC run consisted of two heating and cooling cycles between 0° C. and 290° C. at a rate of 10° C. min⁻¹ with 5 min isothermal holds between each heating and cooling ramp. The glass transition temperature (T_{g}), melting temperature (Tₘ), enthalpy of melting (ΔHₘ), crystallization temperature (T_{c}), temperature of cold crystallization (T_{c}), and enthalpy of cold crystallization (ΔH_{c}) for each sample was determined when applicable with TRIOS software (Universal Analysis). Integration bounds and baselines were determined following the procedure described by Khanna and Kuhn. The following equation was used to calculate percent crystallinity, where ΔHₘ°, the reference enthalpy of melting, is 230.1 J g⁻¹ for nylon-6.

$$\% Crystallinity = ( \frac{Δ {H}_{m} - Δ {H}_{c}}{Δ {H}_{m}^{°}} ) \cdot 100 \%$$

Weight average molar mass (M_{w}), number average molar mass (Mₙ), and dispersity (Ð) values were determined by gel permeation chromatography (GPC) analysis using an Agilent 1260 Infinity II LC system and three Agilent PL HFIPgel 250×4.6 mm columns attached in series with a matching guard column. The column oven was held at 40° C. to reduce backpressure. 1,1,1,3,3,3-Hexafluoroisopropanol with 20 mM of sodium trifluoroacetate was passed through a 0.1 μm filter and used as the mobile phase at a flow rate of 0.35 mL min⁻¹. Samples were prepared in the same solvent at ˜5 mg mL⁻¹, dissolved at room temperature, and passed through 2 μm filters before injection. Detectors consisted of a miniDawn TREOS Multi-Angle Light Scattering detector (Wyatt Technology) used in combination with a Optilab T-Rex Refractive Index detector (Wyatt Technology). Wyatt Technologies Astra Software was used to analyze data, and calculations were performed assuming a dn/dc of 0.2375 for nylon 6 in 1,1,1,3,3,3-hexafluoroisopropanol from the Wyatt Technologies database of dn/dc values.

SEM analysis: SEM analysis was carried out on PA6 films incubated with or without 1 μM NylC_{K}-TS at 60° C. for 10 days. Samples were analyzed by scanning electron microscopy (SEM) on a Hitachi S-4800 High Resolution scanning electron microscope in low- and high-magnification modes at electron voltage=10 kV, current=˜7 amps, and working distance=4 mm. Prior to imaging, the samples were coated with a thin layer (10 nm) of Ir using a

Cressington plasma sputter-coater (208-HR) under inert (Ar) atmosphere and mounted on an aluminum stage with double-sided C and Cu tapes. Image processing was conducted via publicly available software ImageJ2 (NIH).

Chemical synthesis of N-(4-nitrophenyl) butanamide (NB4N): 4-nitroaniline (0.50 g, 3.62 mmol, 1 equiv.) was dissolved in anhydrous dioxane (0.6 mL) and cooled to 0° C. Triethylamine (0.7 mL, 5.03 mmol, 1.4 equiv.) was added dropwise, and then allowed to warm to RT. Butyric anhydride (1.43 g, 9.05 mmol, 2.5 equiv.) in dioxane (0.6 mL), was added dropwise to the reaction, and stirred at RT for 16 h. A saturated aqueous solution of NaHCO₃ (40 mL) was added, and the product was extracted with EtOAc (2×20 mL). The combined organic layers were washed with brine and dried over anhydrous Na₂SO₄, filtered and concentrated to obtain a brown oily residue. The product was precipitated from hexane as a brown solid. ¹H NMR (DMSO-d₆, 400 MHz) δ 10.49 (s, 1H), 8.21 (d, J=9.37 Hz, 2H), 7.48 (d, J=9.37 Hz, 2H), 2.36 (t, J=7.36 Hz, 2H), 1.62 (hex, J=7.36 Hz, 2H), 0.92 (t, J=7.36 Hz, 3H) ppm.

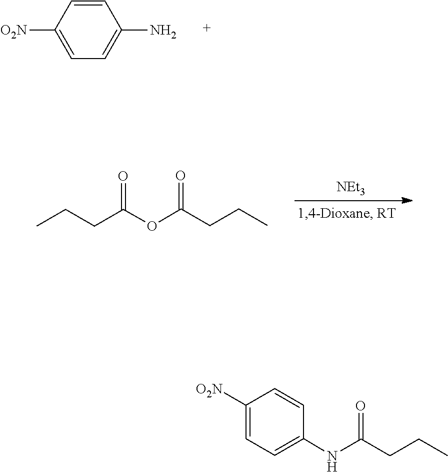

Table 1 summarizes amino acid (aa) and nucleic acid (na) sequences, according to some embodiments of the present disclosure.

TABLE 1
Enzyme Sequences

| SEQ ID | Enzyme | | |
|---|---|---|---|
| NO | Name | Sequence type* | Sequence |
| SEQ ID | NylB′ | aa | NARSTGQHPARYPGAAAGEPTLDSWQEPPHNRWAFAHLGEMVPSAAVSRRPVN |
| NO: 1 | | | APGHALARLGAIAAQLPDLEQRLEQTYTDAFLVLRGTEVVAEYYRAGFAPDDR |
| | | | HLLMSVSKSLCGTVVGALVDEGRIDPAQPVTEYVPELAGSVYDGPSVLQVLDM |
| | | | QISIDYNEDYVDPASEVQTHGRSAGWRTRRHGDPADTYEFLTTLRGDGSTGEF |
| | | | QYCSANTDVLAWIVERVTGLRYVEALSTYLWAKLDADRDATITVDTTGFGFAH |
| | | | GGVSCTARDLARVGRMMLDGGVAPGGRVVSEDWVRRVLAGGSHEAMTDKGFTN |
| | | | TFPDGSYTRQWWCTGNERGNVSGIGIHGQNLWLDPLTDSVIVKLSSWPDPDTE |
| | | | HWHRLQNGILLDVSRALDAV |
| SEQ ID | NylB′ | na | AACGCGCGTTCAACCGGTCAGCATCCTGCCCGCTATCCAGGCGCGGCCGCAGG |
| NO: 2 | | | CGAGCCCACCCTCGATAGTTGGCAGGAACCACCACATAACCGTTGGGCTTTCG |
| | | | CGCATTTGGGTGAAATGGTCCCATCTGCTGCCGTCTCGAGACGTCCGGTTAAC |
| | | | GCTCCGGGACACGCGTTGGCCCGTTTGGGAGCAATTGCCGCCCAGCTGCCGGA |
| | | | CTTGGAACAACGTTTGGAACAGACGTATACAGACGCATTTCTGGTATTACGGG |
| | | | GCACCGAAGTGGTGGCGGAATATTATAGAGCAGGGTTTGCCCCTGACGACCGC |
| | | | CACCTTCTGATGAGCGTGTCGAAATCCCTCTGTGGCACCGTGGTGGGCGCACT |
| | | | CGTCGATGAAGGCCGTATTGATCCGGCACAGCCAGTCACGGAATACGTG |
| | | | CCCGAGCTGGCGGGTAGCGTCTATGATGGACCCTCTGTTTTACAGGTCTTGGA |
| | | | TATGCAGATTTCAATTGATTACAACGAAGACTATGTTGACCCGGCGTCAGAGG |
| | | | TTCAAACTCACGGCCGGTCTGCAGGGTGGCGTACCCGCCGTCATGGGGATCCG |
| | | | GCCGACACGTATGAATTCCTGACCACACTTCGTGGTGATGGCTCCACCGGTGA |
| | | | GTTTCAATATTGCTCCGCCAATACAGATGTGCTGGCCTGGATTGTGGAGCGTG |
| | | | TTACCGGGCTTCGTTATGTAGAAGCCCTGTCAACATATTTGTGGGCCAAACTG |
| | | | GATGCGGATCGTGATGCGACTATCACAGTTGATACCACCGGCTTCGGTTTCGC |
| | | | TCATGGCGGTGTGTCGTGCACAGCACGGGATCTGGCCCGTGTCGGTCGT |
| | | | ATGATGTTAGACGGTGGGGTGGCACCCGGGGGTCGCGTTGTTTCAGAAGACTG |
| | | | GGTCCGTCGTGTTCTGGCTGGCGGATCACATGAAGCAATGACGGACAAGGGCT |
| | | | TTACAAATACGTTTCCCGATGGCTCATATACGCGGCAATGGTGGTGTACAGGC |
| | | | AATGAGCGGGGTAATGTTTCCGGAATAGGTATCCACGGACAGAATTTATGGCT |
| | | | CGATCCATTAACAGACAGCGTCATAGTCAAGTTGTCTTCTTGGCCGGACCCAG |
| | | | ATACTGAGCACTGGCACCGCCTGCAGAATGGCATCCTGCTGGATGTCAGCCGA |
| | | | GCCCTGGATGCTGTT |
| SEQ ID | NylB′- | aa | NARSTGQHPARYPGAAAGEPTLDSWQEPPHNRWAFAHLGEMVPSAAVSRRPVN |
| NO: 3 | SCY | | APGHALARLGAIAAQLPDLEQRLEQTYTDAFLVLRGTEVVAEYYRAGFAPDDR |
| | | | HLLMSVSKSLCGTVVGALVDEGRIDPAQPVTEYVPELAGSVYDGPSVLQVLDM |
| | | | QISIDYNEDYVDPASEVQTHGRSAGWSTRRHGDPADTYEFLTTLRGDGSTGEF |
| | | | QYCSANTDVLAWIVERVTGLRYVEALSTYLWAKLDADRDATITVDTTGFGCAH |
| | | | GGVSCTARDLARVGRMMLDGGVAPGGRVVSEDWVRRVLAGGSHEAMTDKGFTN |
| | | | TFPDGSYTRQWWCTGNERGNVSGIGIHGQNLWLDPLTDSVIVKLSSWPDPYTE |
| | | | HWHRLQNGILLDVSRALDAV |
| SEQ ID | NylB′- | na | AACGCGAGAAGTACCGGCCAGCATCCGGCACGCTATCCGGGTGCGGCCGCAGG |
| NO: 4 | SCY | | GGAACCCACACTGGATAGCTGGCAAGAGCCACCCCATAACCGTTGGGCGTTCG |
| | | | CGCATCTTGGTGAAATGGTACCGTCAGCAGCAGTGTCCCGACGGCCGGTTAAC |
| | | | GCGCCTGGTCATGCGCTGGCACGCCTCGGAGCTATCGCAGCGCAGCTGCCTGA |
| | | | TCTGGAGCAGCGTCTTGAACAGACATATACAGATGCATTTTTGGTACTTCGCG |
| | | | GCACTGAAGTTGTAGCAGAATATTATCGTGCGGGTTTTGCTCCGGACGACCGG |
| | | | CATCTTCTGATGAGTGTTTCTAAATCGTTGTGCGGGACAGTCGTGGGAGCACT |
| | | | CGTCGACGAGGGCAGAATCGATCCTGCACAACCTGTCACTGAATACGTG |
| | | | CCTGAATTAGCCGGATCGGTATATGATGGTCCTTCAGTGCTGCAGGTCCTGGA |
| | | | CATGCAGATTTCGATAGACTACAACGAAGACTATGTTGACCCAGCGAGTGAGG |
| | | | TACAAACTCATGGACGCTCCGCTGGCTGGTCCACACGACGGCACGGTGATCCC |
| | | | GCTGACACTTATGAATTTTTGACAACACTGCGCGGCGATGGTAGTACAGGTGA |
| | | | ATTTCAGTACTGTTCGGCGAATACCGACGTGCTTGCGTGGATCGTGGAACGCG |
| | | | TTACCGGGCTGAGATATGTTGAAGCGCTTTCTACATACCTGTGGGCCAAATTA |
| | | | GACGCCGATCGTGATGCTACAATAACCGTTGACACAACAGGTTTCGGCTGCGC |
| | | | ACATGGTGGTGTTTCGTGTACAGCCCGGGATCTGGCTCGCGTAGGCCGG |
| | | | ATGATGCTGGATGGCGGTGTAGCCCCGGGTGGTCGAGTGGTTTCAGAAGATTG |
| | | | GGTTCGGCGAGTGTTAGCAGGTGGAAGTCACGAAGCTATGACGGATAAAGGCT |
| | | | TTACGAACACCTTCCCTGATGGTTCTTATACACGTCAGTGGTGGTGCACGGGT |
| | | | AACGAACGTGGCAATGTGAGTGGTATCGGAATCCATGGTCAGAACCTGTGGCT |
| | | | CGATCCGCTGACCGATTCGGTTATTGTGAAACTGTCGTCGTGGCCGGACCCGT |
| | | | ACACAGAACATTGGCATAGATTGCAAAACGGTATTCTGCTGGATGTATCACGC |
| | | | GCGTTAGACGCGGTG |
| SEQ ID | NylC_{A} | aa | NTTPVHALTDIDGGIAVDPAPRLAGPPVFGGPGNDAFDLAPVRSTGREMLRED |
| NO: 5 | | | FPGVSIGAAHYEEGPTGATVIHIPAGARTAVDARGGAVGLSGGYDFNHAICLA |
| | | | GGASYGLEAGAGVSGALLERLEYRTGFAEAQLVSSAVIYDESARSTAVYPDKA |
| | | | LGRAALEFAVPGEFPQGRAGAGMSASAGKVDWDRTEITGQGAAFRRLGDVRIL |
| | | | AVVVPNPVGVIMDRAGTVVRGNYDAQTGVRRHPVFDYQEAFAEQVPPVTEAGN |
| | | | TTISAIVTNVRMSPVELNQFAKQVHSSMHRGIQPFHTDMDGDTLFAVTTDEID |
| | | | LPTTPGSSRGRLSVNATALGAIASEVMWDAVLEAGK |
| SEQ ID | NylC_{A} | na | AATACCACACCGGTACACGCTCTTACTGATATTGATGGAGGCATCGCGGTGGA |
| NO: 6 | | | CCCAGCTCCACGTCTTGCAGGACCTCCAGTCTTCGGAGGTCCAGGTAACGACG |
| | | | CTTTTGACCTTGCCCCGGTCCGTTCTACTGGTCGTGAGATGCTGCGCTTCGAT |
| | | | TTCCCGGGTGTTAGCATCGGCGCGGCGCACTATGAAGAAGGTCCGACTGGTGC |
| | | | GACCGTAATTCACATCCCAGCTGGAGCACGTACAGCTGTTGACGCACGTGGTG |
| | | | GTGCAGTGGGTCTGTCCGGTGGCTACGACTTTAATCACGCTATCTGCCTGGCA |
| | | | GGTGGCGCAAGTTATGGTTTAGAAGCTGGCGCAGGTGTGTCTGGTGCGCTGCT |
| | | | GGAACGTTTGGAATATCGTACTGGCTTTGCGGAAGCACAGCTGGTTAGC |
| | | | TCTGCAGTTATTTACGATTTCTCTGCGCGCTCTACCGCAGTCTATCCGGATAA |
| | | | AGCTCTTGGTCGTGCGGCGCTGGAGTTTGCGGTTCCGGGCGAATTCCCGCAGG |
| | | | GTCGTGCTGGTGCGGGCATGAGTGCTTCTGCGGGTAAAGTTGATTGGGACCGT |
| | | | ACTGAAATCACGGGCCAGGGTGCTGCGTTCCGTCGCCTGGGCGACGTTCGCAT |
| | | | CCTGGCCGTAGTTGTCCCGAACCCGGTAGGTGTGATTATGGACCGTGCTGGCA |
| | | | CGGTAGTTCGTGGTAACTACGATGCACAGACTGGCGTTCGTCGTCACCCGGTA |
| | | | TTCGATTACCAGGAAGCTTTTGCGGAGCAGGTTCCTCCGGTTACCGAAGCTGG |
| | | | TAACACCACTATTTCTGCCATCGTTACCAACGTACGCATGTCCCCTGTA |
| | | | GAGCTGAACCAATTCGCAAAACAGGTACACAGCTCTATGCACCGTGGTATCCA |
| | | | GCCTTTCCACACCGACATGGACGGTGACACCCTGTTTGCCGTGACCACTGACG |
| | | | AAATCGATTTGCCGACAACCCCGGGTAGCTCACGTGGTCGCCTGTCTGTAAAC |
| | | | GCTACCGCTCTGGGCGCGATTGCGTCCGAGGTCATGTGGGACGCTGTGCTGGA |
| | | | AGCAGGCAAG |
| SEQ ID | NylC_{A}- | aa | NTTPVHALTDIDGGIAVDPAPRLAGPPVFGGPGNDAFDLAPVRSTGREMLRFD |
| NO: 7 | TS | | FPGVSIGAAHYEEGPTGATVIHIPAGARTAVDARGGAVGLSGGYDENHAICLA |
| | | | GGAGYGLEAGAGVSGALLERLEYRTGFAELQLVSSAVIYDESARSTAVYPDKA |
| | | | LGRAALEFAVPGEFPQGRAGAGMSASAGKVDWDRTEITGQGAAFRRLGDVRIL |
| | | | AVVVPNPVGVIMDRAGTVVRGNYDAQTGVRRHPVFDYQEAFAEQVPPVTQAGN |
| | | | TTISAIVTNVRMSPVELNQFAKQVHSSMHRGIQPFHTDMDGDTLFAVTTDEID |
| | | | LPTTPGSSRGRLSVNATALGAIASEVMWDAVLEAGK |
| SEQ ID | NylC_{A}- | na | AATACCACACCGGTACACGCTCTTACTGATATTGATGGAGGCATCGCGGTGGA |
| NO: 8 | TS | | CCCAGCTCCACGTCTTGCAGGACCTCCAGTCTTCGGAGGTCCAGGTAACGACG |
| | | | CTTTTGACCTTGCCCCGGTCCGTTCTACTGGTCGTGAGATGCTGCGCTTCGAT |
| | | | TTCCCGGGTGTTAGCATCGGCGCGGCGCACTATGAAGAAGGTCCGACTGGTGC |
| | | | GACCGTAATTCACATCCCAGCTGGAGCACGTACAGCTGTTGACGCACGTGGTG |
| | | | GTGCAGTGGGTCTGTCCGGTGGCTACGACTTTAATCACGCTATCTGCCTGGCA |
| | | | GGTGGCGCAGGTTATGGTTTAGAAGCTGGCGCAGGTGTGTCTGGTGCGCTGCT |
| | | | GGAACGTTTGGAATATCGTACTGGCTTTGCGGAACTACAGCTGGTTAGC |
| | | | TCTGCAGTTATTTACGATTTCTCTGCGCGCTCTACCGCAGTCTATCCGGATAA |
| | | | AGCTCTTGGTCGTGCGGCGCTGGAGTTTGCGGTTCCGGGCGAATTCCCGCAGG |
| | | | GTCGTGCTGGTGCGGGCATGAGTGCTTCTGCGGGTAAAGTTGATTGGGACCGT |
| | | | ACTGAAATCACGGGCCAGGGTGCTGCGTTCCGTCGCCTGGGCGACGTTCGCAT |
| | | | CCTGGCCGTAGTTGTCCCGAACCCGGTAGGTGTGATTATGGACCGTGCTGGCA |
| | | | CGGTAGTTCGTGGTAACTACGATGCACAGACTGGCGTTCGTCGTCACCCGGTA |
| | | | TTCGATTACCAGGAAGCTTTTGCGGAGCAGGTTCCTCCGGTTACCCAAGCTGG |
| | | | TAACACCACTATTTCTGCCATCGTTACCAACGTACGCATGTCCCCTGTA |
| | | | GAGCTGAACCAATTCGCAAAACAGGTACACAGCTCTATGCACCGTGGTATCCA |
| | | | GCCTTTCCACACCGACATGGACGGTGACACCCTGTTTGCCGTGACCACTGACG |
| | | | AAATCGATTTGCCGACAACCCCGGGTAGCTCACGTGGTCGCCTGTCTGTAAAC |
| | | | GCTACCGCTCTGGGCGCGATTGCGTCCGAGGTCATGTGGGACGCTGTGCTGGA |
| | | | AGCAGGCAAG |
| SEQ ID | NylCₚ₂ | aa | NTTPVHALTDIDGGIAVDPAPRLAGPPVFGGPGNDAFDLAPVRSTGREMLRFD |
| NO: 9 | | | FPGVSIGAAHYEEGPTGATVIHIPAGARTAVDARGGAVGLSGGYDENHAICLA |
| | | | GGAGYGLEAGAGVSDALLERLEHRTGFAELQLVSSAVIYDESARSTAVYPDKA |
| | | | LGRAALEFAVPGEFPQGRAGAGMSASAGKVDWDRTEITGQGAAFRRLGDVRIL |
| | | | AVVVPNPVGVIVDRAGTVVRGNYDAQTGVRRHPVFDYQEAFAEQVPPVTEAGN |
| | | | TTISAIVTNVRMSPVELNQFAKQVHSSMHRGIQPFHTDMDGDTLFAVTTDEID |
| | | | LPTTPGSSRGRLSVNATALGAIASEVMWDAVLEAGK |
| SEQ ID | NylCₚ₂ | na | AATACCACACCGGTTCACGCGCTGACCGATATCGACGGTGGTATTGCAGTCGA |
| NO: 10 | | | TCCAGCTCCACGTCTAGCTGGTCCTCCAGTTTTCGGTGGTCCGGGCAACGATG |
| | | | CTTTTGACCTGGCACCGGTTCGTAGCACCGGTCGTGAAATGCTGCGTTTTGAT |
| | | | TTCCCGGGCGTCAGCATTGGTGCCGCTCACTATGAAGAAGGTCCGACCGGTGC |
| | | | TACCGTAATTCATATCCCGGCAGGCGCCCGCACTGCCGTTGACGCTCGTGGTG |
| | | | GTGCTGTAGGCCTGTCCGGCGGCTATGATTTTAACCACGCTATTTGTCTGGCT |
| | | | GGCGGCGCAGGTTATGGCCTGGAAGCGGGTGCGGGTGTAAGCGATGCTCTGCT |
| | | | GGAACGTCTGGAGCACCGTACCGGTTTCGCAGAATTACAGCTGGTGTCT |
| | | | TCGGCTGTGATTTACGACTTCTCCGCGCGTTCCACTGCCGTTTACCCGGATAA |
| | | | AGCGCTCGGCCGTGCAGCATTGGAATTCGCGGTACCGGGTGAATTCCCGCAGG |
| | | | GTCGTGCGGGCGCTGGCATGTCCGCTTCCGCTGGTAAGGTAGACTGGGATCGT |
| | | | ACCGAGATCACCGGTCAGGGTGCAGCTTTCCGTAGGCTGGGTGACGTGCGTAT |
| | | | CCTGGCGGTTGTGGTTCCGAATCCTGTGGGTGTTATTGTCGACCGTGCGGGTA |
| | | | CCGTTGTACGCGGTAACTATGATGCTCAGACCGGTGTGCGTCGCCACCCGGTA |
| | | | TTCGACTACCAGGAGGCATTTGCTGAACAGGTTCCGCCAGTCACTGAGGCCGG |
| | | | TAATACCACCATTTCTGCCATCGTTACAAATGTACGTATGTCCCCGGTC |
| | | | GAACTGAATCAGTTCGCTAAACAGGTTCATTCTTCTATGCACCGTGGTATCCA |
| | | | ACCGTTTCACACAGATATGGACGGCGACACCCTGTTTGCTGTAACCACTGATG |
| | | | AAATCGATCTGCCGACCACCCCGGGTTCTTCTCGCGGGCGTCTCTCTGTAAAT |
| | | | GCTACGGCTCTGGGCGCGATTGCGAGCGAAGTAATGTGGGACGCAGTGCTAGA |
| | | | GGCTGGCAAA |
| SEQ ID | NylCₚ₂- | aa | NTTPVHALTDIDGGIAVDPAPRLAGPPVFGGPGNAAFDLAPVRSTGREMLRFD |
| NO: 11 | TS | | FPGVSIGAAHYEEGPTGATVIHIPAGARTAVDARGGAVGLSGGYDENHAICLA |
| | | | GGAGYGLEAGAGVSGALLERLEYRTGFAELQLVSSAVIYDESARSTAVYPDKA |
| | | | LGRAALEFAVPGEFPQGRAGAGMSASAGKVDWDRTEITGQGAAFRRLGDVRIL |
| | | | AVVVPNPVGVIMDRAGTVVRGNYDAQTGVRRHPVFDYQEAFAEQVPPVTQAGN |
| | | | TTISAIVTNVRMSPVELNQFAKQVHSSMHRGIQPFHTDMDGDTLFAVTTDEID |
| | | | LPTTPGSSRGRLSVNATALGAIASEVMWDAVLEAGK |
| SEQ ID | NylCₚ₂- | na | AATACCACACCGGTTCACGCGCTGACCGATATCGACGGTGGTATTGCAGTCGA |
| NO: 12 | TS | | TCCAGCTCCACGTCTAGCTGGTCCTCCAGTTTTCGGTGGTCCGGGCAACGCTG |
| | | | CTTTTGACCTGGCACCGGTTCGTAGCACCGGTCGTGAAATGCTGCGTTTTGAT |
| | | | TTCCCGGGCGTCAGCATTGGTGCCGCTCACTATGAAGAAGGTCCGACCGGTGC |
| | | | TACCGTAATTCATATCCCGGCAGGCGCCCGCACTGCCGTTGACGCTCGTGGTG |
| | | | GTGCTGTAGGCCTGTCCGGCGGCTATGATTTTAACCACGCTATTTGTCTGGCT |
| | | | GGCGGCGCAGGTTATGGCCTGGAAGCGGGTGCGGGTGTAAGCGGTGCTCTGCT |
| | | | GGAACGTCTGGAGTACCGTACCGGTTTCGCAGAATTACAGCTGGTGTCT |
| | | | TCGGCTGTGATTTACGACTTCTCCGCGCGTTCCACTGCCGTTTACCCGGATAA |
| | | | AGCGCTCGGCCGTGCAGCATTGGAATTCGCGGTACCGGGTGAATTCCCGCAGG |
| | | | GTCGTGCGGGCGCTGGCATGTCCGCTTCCGCTGGTAAGGTAGACTGGGATCGT |
| | | | ACCGAGATCACCGGTCAGGGTGCAGCTTTCCGTAGGCTGGGTGACGTGCGTAT |
| | | | CCTGGCGGTTGTGGTTCCGAATCCTGTGGGTGTTATTATGGACCGTGCGGGTA |
| | | | CCGTTGTACGCGGTAACTATGATGCTCAGACCGGTGTGCGTCGCCACCCGGTA |
| | | | TTCGACTACCAGGAGGCATTTGCTGAACAGGTTCCGCCAGTCACTCAGGCCGG |
| | | | TAATACCACCATTTCTGCCATCGTTACAAATGTACGTATGTCCCCGGTC |
| | | | GAACTGAATCAGTTCGCTAAACAGGTTCATTCTTCTATGCACCGTGGTATCCA |
| | | | ACCGTTTCACACAGATATGGACGGCGACACCCTGTTTGCTGTAACCACTGATG |
| | | | AAATCGATCTGCCGACCACCCCGGGTTCTTCTCGCGGGCGTCTCTCTGTAAAT |
| | | | GCTACGGCTCTGGGCGCGATTGCGAGCGAAGTAATGTGGGACGCAGTGCTAGA |
| | | | GGCTGGCAAA |
| SEQ ID | NylC_{K} | aa | NTTPVHALTDIDGGIAVDPAPRLAGPPVFGGPGNAAFDLVPVRSTGRETLRED |
| NO: 13 | | | FPGVSVGSAHYEEGPTGATVIHIPAGARTAVDARGGAVGLSGGYDFNHAICLA |
| | | | GGASYGLEAGAGVSGALLERLEYRTGFAEAQLVSSAVIYDESARSTAVYPDKA |
| | | | LGRAALEFAVPGEFPQGRAGAGMSASAGKVDWDRTEITGQGAAFRRLGDVRIL |
| | | | AVVVPNPVGVIMDRAGGIVRGNYDAQTGVRRHPVFDYQEAFAEQLPPVTQAGN |
| | | | TTISAIVTNVRMSPVELNQFAKQVHSSMHRGIQPFHTDMDGDTLFAVTTDEID |
| | | | LPTTPGSSRGRLSVNATALGAIASEVMWDAVLEAAK |
| SEQ ID | NylC_{K} | na | AATACCACACCGGTGCATGCTCTCACCGATATCGACGGTGGCATTGCGGTAGA |
| NO: 14 | | | TCCAGCTCCACGTCTTGCAGGTCCACCTGTGTTCGGTGGACCTGGTAACGCTG |
| | | | CATTCGATCTGGTTCCGGTACGTTCCACCGGTCGTGAAACCCTGCGTTTCGAT |
| | | | TTTCCGGGGGTTTCTGTGGGTAGCGCGCACTACGAAGAGGGTCCGACTGGCGC |
| | | | CACCGTCATCCACATCCCGGCTGGGGCACGTACTGCTGTAGATGCACGTGGCG |
| | | | GCGCAGTGGGCCTGTCCGGTGGCTACGACTTCAACCACGCTATTTGTCTGGCT |
| | | | GGTGGTGCTTCTTACGGCCTGGAGGCTGGCGCAGGCGTTTCCGGTGCTCTGCT |
| | | | GGAACGCCTCGAATACCGCACAGGTTTCGCTGAAGCCCAACTGGTGTCT |
| | | | TCCGCAGTAATCTACGACTTCTCTGCCCGTTCAACCGCAGTGTATCCAGACAA |
| | | | AGCCTTGGGTCGTGCTGCGCTGGAATTTGCCGTACCGGGTGAATTCCCACAGG |
| | | | GTCGCGCTGGTGCCGGCATGAGCGCCAGCGCGGGCAAAGTTGATTGGGATCGT |
| | | | ACCGAGATCACAGGTCAGGGCGCTGCTTTCCGCCGTCTGGGCGATGTGCGTAT |
| | | | CCTGGCTGTTGTTGTGCCAAACCCAGTTGGCGTAATTATGGATCGTGCAGGCG |
| | | | GTATCGTCCGTGGCAACTATGATGCGCAAACTGGCGTTCGCCGTCACCCAGTA |
| | | | TTTGATTACCAGGAAGCTTTCGCGGAACAACTGCCGCCTGTTACTCAGGCGGG |
| | | | CAACACGACCATCAGCGCGATCGTTACCAATGTCCGCATGAGTCCGGTG |
| | | | GAACTGAATCAATTCGCCAAACAGGTGCATAGCTCTATGCACCGCGGTATCCA |
| | | | GCCATTTCACACCGATATGGACGGCGATACCCTGTTCGCCGTGACTACCGACG |
| | | | AAATCGATCTCCCGACTACGCCGGGCTCCTCCCGTGGCCGCCTGTCTGTTAAC |
| | | | GCAACCGCTCTGGGCGCAATTGCTTCTGAAGTTATGTGGGACGCCGTATTAGA |
| | | | AGCGGCGAAG |
| SEQ ID | NylC_{K}- | aa | NTTPVHALTDIDGGIAVDPAPRLAGPPVFGGPGNAAFDLVPVRSTGRETLRED |
| NO: 15 | TS | | FPGVSVGSAHYEEGPTGATVIHIPAGARTAVDARGGAVGLSGGYDFNHAICLA |
| | | | GGAGYGLEAGAGVSGALLERLEYRTGFAELQLVSSAVIYDESARSTAVYPDKA |
| | | | LGRAALEFAVPGEFPQGRAGAGMSASAGKVDWDRTEITGQGAAFRRLGDVRIL |
| | | | AVVVPNPVGVIMDRAGGIVRGNYDAQTGVRRHPVFDYQEAFAEQLPPVTQAGN |
| | | | TTISAIVTNVRMSPVELNQFAKQVHSSMHRGIQPFHTDMDGDTLFAVTTDEID |
| | | | LPTTPGSSRGRLSVNATALGAIASEVMWDAVLEAAK |
| SEQ ID | NylC_{K}- | na | AATACCACACCGGTGCATGCTCTCACCGATATCGACGGTGGCATTGCGGTAGA |
| NO: 16 | TS | | TCCAGCTCCACGTCTTGCAGGTCCACCTGTGTTCGGTGGACCTGGTAACGCTG |
| | | | CATTCGATCTGGTTCCGGTACGTTCCACCGGTCGTGAAACCCTGCGTTTCGAT |
| | | | TTTCCGGGGGTTTCTGTGGGTAGCGCGCACTACGAAGAGGGTCCGACTGGCGC |
| | | | CACCGTCATCCACATCCCGGCTGGGGCACGTACTGCTGTAGATGCACGTGGCG |
| | | | GCGCAGTGGGCCTGTCCGGTGGCTACGACTTCAACCACGCTATTTGTCTGGCT |
| | | | GGTGGTGCTGGTTACGGCCTGGAGGCTGGCGCAGGCGTTTCCGGTGCTCTGCT |
| | | | GGAACGCCTCGAATACCGCACAGGTTTCGCTGAACTCCAACTGGTGTCT |
| | | | TCCGCAGTAATCTACGACTTCTCTGCCCGTTCAACCGCAGTGTATCCAGACAA |
| | | | AGCCTTGGGTCGTGCTGCGCTGGAATTTGCCGTACCGGGTGAATTCCCACAGG |
| | | | GTCGCGCTGGTGCCGGCATGAGCGCCAGCGCGGGCAAAGTTGATTGGGATCGT |
| | | | ACCGAGATCACAGGTCAGGGCGCTGCTTTCCGCCGTCTGGGCGATGTGCGTAT |
| | | | CCTGGCTGTTGTTGTGCCAAACCCAGTTGGCGTAATTATGGATCGTGCAGGCG |
| | | | GTATCGTCCGTGGCAACTATGATGCGCAAACTGGCGTTCGCCGTCACCCAGTA |
| | | | TTTGATTACCAGGAAGCTTTCGCGGAACAACTGCCGCCTGTTACTCAGGCGGG |
| | | | CAACACGACCATCAGCGCGATCGTTACCAATGTCCGCATGAGTCCGGTG |
| | | | GAACTGAATCAATTCGCCAAACAGGTGCATAGCTCTATGCACCGCGGTATCCA |
| | | | GCCATTTCACACCGATATGGACGGCGATACCCTGTTCGCCGTGACTACCGACG |
| | | | AAATCGATCTCCCGACTACGCCGGGCTCCTCCCGTGGCCGCCTGTCTGTTAAC |
| | | | GCAACCGCTCTGGGCGCAATTGCTTCTGAAGTTATGTGGGACGCCGTATTAGA |
| | | | AGCGGCGAAG |

*aa = amino acid; na = nucleic acid

### EXAMPLES

Example 1. A method comprising hydrolyzing a polyamide by contacting the polyamide with a hydrolase.

Example 2. The method of Example 1, wherein the polyamide comprises nylon-6, nylon 6,6, or a combination thereof.

Example 3. The method of either Example 1 and/or Example 2, wherein the hydrolyzing produces 6-aminohexanoic acid, an oligomer of 6-aminohexanoic acid, ε-caprolactam, or combinations thereof.

Example 4. The method of any one of Examples 1-3, wherein the oligomer is a compound of at least one of Structure (I) or Structure (II):

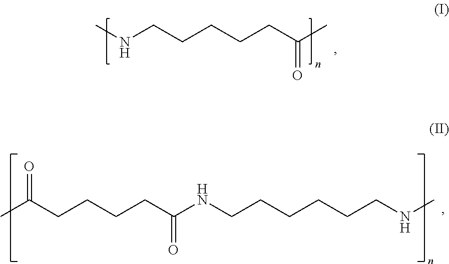

wherein n is between 2 and 5.

Example 5. The method any one of Examples 1-4, wherein the hydrolase is thermostable.

Example 6. The method any one of Examples 1-5, wherein the hydrolase is selected from an SHD-hydrolase, an NylB-type hydrolase, an NylC-type hydrolase, and combinations thereof.

Example 7. The method any one of Examples 1-6, wherein the hydrolase comprises an amino acid sequence that is at least 90%, 95%, 96%, 97%, 98%, or 99% identical to SEQ ID NO: 1.

Example 8. The method any one of Examples 1-7, wherein the hydrolase comprises an amino acid sequence according to SEQ ID NO: 1 having an R-S mutation at residue 186, an F-C mutation at residue 263, a D-Y mutation at residue 369, or any combination thereof.

Example 9. The method any one of Examples 1-8, wherein the hydrolase comprises a nucleic acid sequence that is at least 90%, 95%, 96%, 97%, 98%, or 99% identical to SEQ ID NO: 2.

Example 10. The method any one of Examples 1-9, wherein the hydrolase comprises an amino acid sequence that is at least 99% identical to SEQ ID NO: 3 (NylB′-SCY; see spreadsheet).

Example 11. The method any one of Examples 1-10, wherein the hydrolase comprises an amino acid sequence that is at least 90%, 95%, 96%, 97%, 98%, or 99% identical to SEQ ID NO: 5.

Example 12. The method any one of Examples 1-11, wherein the hydrolase comprises an amino acid sequence according to SEQ ID NO: 5 having an S-G mutation at residue 110, an A-L mutation at residue 136, an E-Q mutation at residue 262, or any combination thereof.

Example 13. The method any one of Examples 1-12, wherein the hydrolase comprises a nucleic acid sequence that is at least 90%, 95%, 96%, 97%, 98%, or 99% identical to SEQ ID NO: 6.

Example 14. The method any one of Examples 1-13, wherein the hydrolase comprises an amino acid sequence that is at least 99% identical to SEQ ID NO: 7.

Example 15. The method any one of Examples 1-14, wherein the hydrolase comprises an amino acid sequence that is at least 90%, 95%, 96%, 97%, 98%, or 99% identical to SEQ ID NO: 9.

Example 16. The method any one of Examples 1-15, wherein the hydrolase comprises an amino acid sequence according to SEQ ID NO: 9 having a D-A mutation at residue 35, a D-G mutation at residue 121, an H-Y mutation at residue 129, a V-M mutation at residue 224, an E-Q mutation at residue 262, or any combination thereof.

Example 17. The method any one of Examples 1-16, wherein the hydrolase comprises a nucleic acid sequence that is at least 90%, 95%, 96%, 97%, 98%, or 99% identical to SEQ ID NO: 10.

Example 18. The method any one of Examples 1-17, wherein the hydrolase comprises an amino acid sequence that is at least 99% identical to SEQ ID NO: 11.

Example 19. The method any one of Examples 1-18, wherein the hydrolase comprises an amino acid sequence that is at least 90%, 95%, 96%, 97%, 98%, or 99% identical to SEQ ID NO: 13.

Example 20. The method any one of Examples 1-19, wherein the hydrolase comprises an amino acid sequence according to SEQ ID NO: 13 having a S-G mutation at residue 110, a A-L mutation at residue 136, or any combination thereof.

Example 21. The method any one of Examples 1-20, wherein the hydrolase comprises a nucleic acid sequence that is at least 90%, 95%, 96%, 97%, 98%, or 99% identical to SEQ ID NO: 14.

Example 22. The method any one of Examples 1-21, wherein the hydrolase comprises an amino acid sequence that is at least 99% identical to SEQ ID NO: 15.

Example 23. The method any one of Examples 1-22, wherein the contacting is performed in an aqueous solution.

Example 24. The method any one of Examples 1-23, wherein the contacting is performed at a pH between 5 and 10 or between 7 and 8.

Example 25. The method any one of Examples 1-24, wherein the contacting is performed at a temperature between 22° C. and 100° C. or between 40° C. and 70° C.

Example 26. The method any one of Examples 1-25, wherein the contacting is performed at a temperature above 48° C., or above 49° C., or above 50° C., or above 51° C.

Example 27. The method any one of Examples 1-26, wherein the contacting is performed for a period of time between 1 hour and 300 hours, or between 1 hour and 24 hours, or between 1 hour and 10 hours.

Example 28. The method any one of Examples 1-27, wherein the hydrolase is present in the aqueous solution at a concentration between 0.01 μM and 100 μM, or between 0.01 μM and 50 μM, or between 0.01 μM and 10 μM.

Example 29. The method any one of Examples 1-283, wherein the hydrolase and the polyamide are present in the aqueous solution at a hydrolase to polyamide ratio between 0.01 mM/g and 100 mM/g, or between 0.1 mM/g and 10 mM/g, or between 0.08 mM/g and 0.77 mM/g.

Example 30. The method any one of Examples 1-29, wherein the polyamide is present in the aqueous solution at a concentration between 0.01 wt % and 10 wt % or between 0.32 wt % and 1.6 wt %.

Example 31. The method any one of Examples 1-30, wherein the aqueous solution further comprises bovine serum albumin (BSA).

Example 32. The method any one of Examples 1-31, wherein the BSA is present at a concentration between 0.01 μM and 10 μM or between 0.5 μM and 2 μM.

Example 33. The method any one of Examples 1-32, wherein the aqueous solution further comprises a buffer.

Example 34. The method any one of Examples 1-33, wherein the buffer comprises sodium phosphate.

Example 35. The method any one of Examples 1-34, wherein the buffer is present in the aqueous solution at a concentration between 1 mM and 1000 mM or between 100 mM and 150 mM.

Example 36. The method any one of Examples 1-35, wherein the aqueous solution further comprises a salt.

Example 37. The method any one of Examples 1-36, wherein the salt comprises NaCl.

Example 38. The method any one of Examples 1-37 wherein the salt is present in the aqueous solution at a concentration between 100 mM and 200 mM.

Example 39. The method any one of Examples 1-38, further comprising contacting the polyamide with a solvent.

Example 40. The method any one of Examples 1-39, wherein the solvent contacting is performed before the hydrolase contacting.

Example 41. The method any one of Examples 1-40, wherein the solvent contacting is performed during the hydrolase contacting.

Example 42. The method any one of Examples 1-41, wherein the solvent comprises a phenol.

Example 43. The method any one of Examples 1-42, wherein the solvent comprises phenol, guiacol, 4-ethyl guiacol, 4-propyl guiacol, 2-iso-propylphenol, 4-propylphenol, or any combination thereof.

Example 44. The method any one of Examples 1-43, further comprising, prior to the contacting, converting the polyamide from a first shape to a second shape, wherein: the first shape comprises a layer, the second shape comprises a particle, and the particle has a particle size between 5 μM and 50 μM.

Example 45. The method any one of Examples 1-44, wherein the polyamide has a crystallinity between 13.2% and 47.6%.

As used herein the term “substantially” is used to indicate that exact values are not necessarily attainable. By way of example, one of ordinary skill in the art will understand that in some chemical reactions 100% conversion of a reactant is possible, yet unlikely. Most of a reactant may be converted to a product and conversion of the reactant may asymptotically approach 100% conversion. So, although from a practical perspective 100% of the reactant is converted, from a technical perspective, a small and sometimes difficult to define amount remains. For this example of a chemical reactant, that amount may be relatively easily defined by the detection limits of the instrument used to test for it. However, in many cases, this amount may not be easily defined, hence the use of the term “substantially”. In some embodiments of the present invention, the term “substantially” is defined as approaching a specific numeric value or target to within 20%, 15%, 10%, 5%, or within 1% of the value or target. In further embodiments of the present invention, the term “substantially” is defined as approaching a specific numeric value or target to within 1%, 0.9%, 0.8%, 0.7%, 0.6%, 0.5%, 0.4%, 0.3%, 0.2%, or 0.1% of the value or target.

As used herein, the term “about” is used to indicate that exact values are not necessarily attainable. Therefore, the term “about” is used to indicate this uncertainty limit. In some embodiments of the present invention, the term “about” is used to indicate an uncertainty limit of less than or equal to ±20%, ±15%, ±10%, ±5%, or ±1% of a specific numeric value or target. In some embodiments of the present invention, the term “about” is used to indicate an uncertainty limit of less than or equal to ±1%, ±0.9%, ±0.8%, ±0.7%, ±0.6%, ±0.5%, ±0.4%, ±0.3%, ±0.2%, or ±0.1% of a specific numeric value or target.

The foregoing discussion and examples have been presented for purposes of illustration and description. The foregoing is not intended to limit the aspects, embodiments, or configurations to the form or forms disclosed herein. In the foregoing Detailed Description for example, various features of the aspects, embodiments, or configurations are grouped together in one or more embodiments, configurations, or aspects for the purpose of streamlining the disclosure. The features of the aspects, embodiments, or configurations, may be combined in alternate aspects, embodiments, or configurations other than those discussed above. This method of disclosure is not to be interpreted as reflecting an intention that the aspects, embodiments, or configurations require more features than are expressly recited in each claim. Rather, as the following claims reflect, inventive aspects lie in less than all features of a single foregoing disclosed embodiment, configuration, or aspect. While certain aspects of conventional technology have been discussed to facilitate disclosure of some embodiments of the present invention, the Applicants in no way disclaim these technical aspects, and it is contemplated that the claimed invention may encompass one or more of the conventional technical aspects discussed herein. Thus, the following claims are hereby incorporated into this Detailed Description, with each claim standing on its own as a separate aspect, embodiment, or configuration.

What is claimed is:

1. A method comprising hydrolyzing a polyamide by contacting the polyamide with a hydrolase.

2. The method of claim 1, wherein the polyamide comprises nylon-6, nylon 6,6, or a combination thereof.

3. The method of claim 1, wherein the hydrolyzing produces 6-aminohexanoic acid, an oligomer of 6-aminohexanoic acid, ε-caprolactam, or combinations thereof.

4. The method of claim 3, wherein the oligomer is a compound of at least one of Structure (I) or Structure (II): 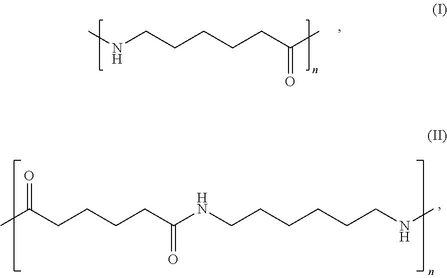
wherein n is between 2 and 5.

5. The method of claim 1, wherein the hydrolase is selected from an SHD-hydrolase, an NylB-type hydrolase, an NylC-type hydrolase, and combinations thereof.

6. The method of claim 5, wherein the hydrolase comprises an amino acid sequence that is at least 90% identical to SEQ ID NO: 1.

7. The method of claim 5, wherein the hydrolase comprises an amino acid sequence according to SEQ ID NO: 1 having an R-S mutation at residue 186, an F-C mutation at residue 263, a D-Y mutation at residue 369, or any combination thereof.

8. The method of claim 5, wherein the hydrolase comprises a nucleic acid sequence that is at least 90% identical to SEQ ID NO: 2.

9. The method of claim 5, wherein the hydrolase comprises an amino acid sequence that is at least 90% identical to SEQ ID NO: 5.

10. The method of claim 5, wherein the hydrolase comprises an amino acid sequence according to SEQ ID NO: 5 having an S-G mutation at residue 110, an A-L mutation at residue 136, an E-Q mutation at residue 262, or any combination thereof.

11. The method of claim 5, wherein the hydrolase comprises a nucleic acid sequence that is at least 90% identical to SEQ ID NO: 6.

12. The method of claim 5, wherein the hydrolase comprises an amino acid sequence that is at least 90% identical to SEQ ID NO: 9.

13. The method of claim 5, wherein the hydrolase comprises an amino acid sequence according to SEQ ID NO: 9 having a D-A mutation at residue 35, a D-G mutation at residue 121, an H-Y mutation at residue 129, a V-M mutation at residue 224, an E-Q mutation at residue 262, or any combination thereof.

14. The method of claim 5, wherein the hydrolase comprises a nucleic acid sequence that is at least 90% identical to SEQ ID NO: 10.

15. The method of claim 5, wherein the hydrolase comprises an amino acid sequence that is at least 90% identical to SEQ ID NO: 13.

16. The method of claim 5, wherein the hydrolase comprises an amino acid sequence according to SEQ ID NO: 13 having a S-G mutation at residue 110, a A-L mutation at residue 136, or any combination thereof.

17. The method of claim 5, wherein the hydrolase comprises a nucleic acid sequence that is at least 90% identical to SEQ ID NO: 14.

18. The method of claim 1, wherein the contacting is performed in an aqueous solution.

19. The method of claim 1. wherein the contacting is performed at a temperature between 22° C. and 100° C.

20. The method of claim 18, wherein the aqueous solution further comprises bovine serum albumin (BSA).
